(19) United States
(12) Patent Application Publication
(10) Pub. No.: US 2018/0106806 A1
(43) Pub. Date: Apr. 19, 2018

(54) Tumor Analytical Methods

(71) Applicant: REGENTS OF THE UNIVERSITY OF MINNESOTA, Minneapolis, MN (US)

(72) Inventors: Milcah C. Scott, Minneapolis, MN (US); Aaron L. Sarver, Minneapolis, MN (US); Jaime F. Modiano, St. Paul, MN (US); Subbaya Subramanian, Minneapolis, MN (US); David A. Largaespada, Mounds View, MN (US); Logan G. Spector, Minneapolis, MN (US)

(21) Appl. No.: 15/783,352

(22) Filed: Oct. 13, 2017

Related U.S. Application Data
(60) Provisional application No. 62/407,841, filed on Oct. 13, 2016.

(51) Int. Cl. G01N 33/574; G06F 19/12; G06F 19/24

(52) U.S. Cl. CPC G01N 33/57407; G06F 19/12; G01N 2800/7028; G01N 2800/56; G06F 19/24

(57) ABSTRACT

A method for detecting aggressive tumor behavior and/or increased risk for tumor metastasis generally includes analyzing a tumor sample from the subject for expression of transcripts from coding regions of the cell cycle gene cluster, the immune-1 gene cluster, or the immune-2 gene cluster; computing a sum of log₂-transformed mean-centered expression values, thereby generating a Gene Cluster Expression Summary Score (GCESS) for the sample; and detecting a tumor with aggressive behavior and/or increased risk for tumor metastasis. An aggressive tumor and/or increased risk for tumor metastasis may be indicated where the cell cycle gene cluster is analyzed and the sample GCESS is greater than 0, or the immune-1 gene cluster or the immune-2 gene cluster is analyzed and the sample GCESS is less than 0.

### CROSS-REFERENCE TO RELATED APPLICATION

This application claims priority to U.S. Provisional Patent Application No. 62/407,841, filed Oct. 13, 2016, which is incorporated herein by reference.

### GOVERNMENT FUNDING

This invention was made with government support under R01 CA113636 awarded by the National Institutes of Health, P30 CA077598 awarded by the National Institutes of Health, R50 CA211249 awarded by the National Institutes of Health, R21 CA208529 awarded by the National Institutes of Health, and T32 CA099936, awarded by the National Institutes of Health. The government has certain rights in the invention.

### SUMMARY

This disclosure describes, in one aspect, a method for detecting aggressive tumor behavior. Generally, the method includes analyzing a tumor sample from the subject for expression of transcripts from coding regions of the cell cycle gene cluster, the immune-1 gene cluster, or the immune-2 gene cluster; computing a sum of log₂-transformed mean-centered expression values, thereby generating a Gene Cluster Expression Summary Score (GCESS) for the sample; and detecting a tumor with aggressive behavior. An aggressive tumor may be indicated where the cell cycle gene cluster is analyzed and the sample GCESS is greater than 0, or the immune-1 gene cluster or the immune-2 gene cluster is analyzed and the sample GCESS is less than 0.

In another aspect, this disclosure describes a method for detecting relative risk of metastasis in a patient diagnosed with a tumor. Generally, the method includes analyzing a tumor sample from the patient for expression of transcripts from coding regions of the cell cycle gene cluster, the immune-1 gene cluster, or the immune-2 gene cluster, computing a sum of log 2-transformed mean-centered expression values, thereby generating a Gene Cluster Expression Summary Score (GCESS) for the sample, and detecting above average risk of metastasis. An above average risk of metastasis can be indicated where the cell cycle gene cluster is analyzed and the sample GCESS is greater than 0, or the immune-1 gene cluster or the immune-2 gene cluster is analyzed and the sample GCESS is less than 0.

In either aspect, the immune-1 gene cluster and the immune-2 gene cluster can be analyzed and the sample GCESS is less than 0.

In either aspect, aggressive tumor behavior can include rapid local tumor growth, rapid progression to metastasis, and/or below average response to standard of care treatment.

In either aspect, the tumor can include osteosarcoma (OS), include cervical squamous cell carcinoma and endocervical adenocarcinoma (CESC), colon adenocarcinoma (COAD), glioblastoma (GBM), acute myeloid leukemia (LAML), prostate cancer (PRAD), thyroid (THCA) cancer, bladder urothelial carcinoma (BLCA), breast invasive carcinoma (BRCA), head and neck squamous cell carcinoma (HNSC), kidney renal clear cell carcinoma (KIRC), liver hepatocellular carcinoma (LIHC), lung adenocarcinoma (LUAD), lung squamous cell carcinoma (LUSC), ovarian serous cystadenocarcinoma (OV), pancreatic adenocarcinoma (PAAD), rectum adenocarcinoma (READ), and skin cutaneous melanoma (SKCM).

In the first aspect, the method can further include administering to the subject treatment for a tumor showing aggressive behavior.

In the second aspect, the method can further include administering to the subject treatment for a metastatic tumor.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Osteosarcoma (OS) transcriptome profiles across three species are more similar to each other than to other types of human tumors. Pairwise Pearson correlation coefficients were calculated using 12,062 genes common between human osteosarcoma (HOS), dog osteosarcoma (DOS), mouse osteosarcoma, and TCGA data for other human cancers. 25 samples from RNA-Seq expression data were randomly selected for TCGA primary tumors from Cervical Squamous Cell Carcinoma and Endocervical Adenocarcinoma (CESC), Colon Adenocarcinoma (COAD), Glioblastoma (GBM), Acute Myeloid Leukemia (LAML), Prostate (PRAD), and Thyroid (THCA) cancer data sets. HOS is the human osteosarcoma samples sequenced in this study. HOS2(21) and HOS3(16) are previously published independent cohorts. HOS2 was mapped using the same protocol as HOS while HOS3 was mapped using the TCGA RNA-Seq protocol. Correlations were calculated between each data set and the combined (A) Human (B) Dog and (C) Mouse osteosarcoma data sets.

FIG. 2. Osteosarcoma transcriptome profiles show common tumor heterogeneity across human, mouse, and dog samples. Tumor samples and cell line data (indicated by blue arrows) were included for each species. FPKM values were log transformed and mean centered within each species. Invariant genes were then removed leaving (A) human (n=9,190), (B) mouse (n=8,051), and (C) dog (n=8,003) genes which were used for unsupervised average linkage clustering. Transcripts with increased levels are shown in yellow while transcripts with decreased levels are shown in blue. Gene clusters with correlation >0.60 and containing ≥60 genes were assigned a number and are indicated by bars to the right of each heat map. Gene clusters common to more than one species are indicated by symbols listed below. (D) To identify whether any of the clusters systematically identified were present in multiple species the number of overlapping genes in each cluster was calculated. Of the five commonly present gene clusters, a cluster with annotations to cell cycle/mitosis (indicated by *) and two clusters annotated to immune cell functions (indicated by ^{Φ}) were present in all three species. Additionally, a cluster of genes associated with muscle differentiation (indicated by ^{χ}) as well as a cluster with no strong functional associations (unknown) was present in both human and mouse tumors.

FIG. 3. Gene Cluster Expression Summary Scores (GCESSs) represent the relative amount of transcript present for each cluster of genes. (A) To generate a GCESS, the log 2-transformed and mean-centered values for each gene in a cluster are summed to generate a single score (GCESS value) for each sample. Samples with high relative expression levels have large positive GCESSs while samples with low relative levels have large negative GCESSs. GCESSs for human (tumors, normal bone, osteosarcoma cell lines), mouse (tumors, osteosarcoma cell lines), and dog (tumors, osteoblasts, osteosarcoma cell lines) samples were calculated and plotted for (B) the cell cycle/mitosis cluster, (C) the immune cell cluster-1, and (D) the immune cell cluster-2, and (E) the muscle differentiation cluster.

FIG. 4. Correlation between high cell cycle or low immune cell GCESSs and poor survival. Samples were ranked by GCESS and divided into quartiles groups (Q1=lowest GCESSs, Q4=highest GCESSs). KM analyses were performed to determine correlations between low/high GCESSs and survival. (A) A significant association with poor survival was observed with a high cell cycle GCESS in the dog cohort, and a strong trend was also present in the human data. In the human data a low immune cell GCESS was significantly associated with a worse survival outcome. KM curves show worse outcomes for i) ii) iii) iv) (B) Low immune cell GCESS was associated with increased presence of metastasis at diagnosis. In the HOS2 cohort, 17/35 patients (49%) presented with metastasis at diagnosis. The immune cell GCESSs (shown with a circle) were plotted on the Y-axis and the relative rank positions, from low to high, were plotted on the X-axis. Patients with metastasis at diagnosis are colored in red (C) Fisher's exact test shows that significantly more patients with low immune cell GCESS presented with metastasis than those with a high immune cell GCESS.

FIG. 5. Replication of association between high cell cycle GCESS or low immune cell GCESS and poor survival outcomes using array data from independent human osteosarcoma cohort. Series GSE21257, which consisted of genome-wide expression data of 53 human osteosarcoma tumors produced from the Illumina human-6 v2.0 expression array was downloaded. Patient outcome data, including survival and metastasis, was included. (A) Following a systematic strategy similar to the one used for the RNA-Seq data, 14 strong and highly correlated clusters were identified. (B) Gene overlap analyses comparing clusters derived from array and RNA-Seq data sets identified four array clusters which corresponded to the RNA-Seq immune clusters (indicated by ^{Φ}), and two array gene clusters which corresponded to the RNA-Seq cell cycle gene cluster (indicated by *). (C) KM-analyses utilizing GCESSs generated from each of these array clusters showed that patients whose tumors had low immune cell GCESSs were more likely to succumb to osteosarcoma. This result was independently observable for each of the four immune-cell-related gene clusters from the Illumina array data. KM-analyses using time till metastases showed that patients with low immune cell GCESSs were more likely to develop metastases. Additionally, patients with high cell cycle GCESSs were more likely to develop metastases.

FIG. 6. Model depicting how immune cell components may be preventing the occurrence of metastasis in osteosarcoma patients.

FIG. 7. Cell cycle GCESS (x-axis) plotted against immune cell GCESS (y-axis).

FIG. 8. GCESSs were calculated for each of the TCGA tumor datasets (using the genes comprising the respective human osteosarcoma clusters to identify each tumor's cell cycle and immune cell clusters) and KM-analyses were used to determine correlation with poor survival outcomes. (A) High cell cycle GCESSs (based on human osteosarcoma cluster-4) were significantly associated (in red) with poor outcomes in KIRC, LIHC, LUAD, and PAAD. (B) Low immune cell GCESSs (based on human osteosarcoma cluster-1 (Monocyte enriched)) were significantly associated (in grey) with poor outcomes in CESC, COAD, LUAD, and SKCM. (C) Low immune cell GCESSs (based on human osteosarcoma cluster-8 (T-cell enriched)) were significantly associated (in grey) with poor outcomes in BRCA, CESC, COAD, HNSC, LIHC, LUAD, rectum adenocarcinoma (READ), and SKCM.

FIG. 9. OS transcriptome profiles show common inter-tumor transcriptional variation across human, mouse, and dog samples. (A) FPKM values derived from OS tumors and cell line data (indicated by black bars below heatmaps) were log transformed and mean centered within each species. Invariant genes were then removed leaving (i) human (n=9,190), (ii) mouse (n=8,051), and (iii) dog (n=8,003) genes which were used for unsupervised average linkage clustering. Transcripts with increased levels are shown in yellow while transcripts with decreased levels are shown in blue. Transcript level clusters with correlation >0.60 and containing ≥60 genes were systematically identified and these clusters are visualized with a numbered black bar to the right of each of the heatmaps. Lists of each gene in each cluster are provided in FIG. 12. Gene clusters observed in more than one species are surrounded by colored boxes and given a reference number. The “Cell Cycle” conserved transcript cluster is shown in red (HOS-4, MOS-8, DOS-3). The “Immune-1” transcript cluster is shown in green (HOS-1, MOS-4, DOS-4). The “Immune-2” transcript cluster is shown in purple (HOS-8, MOS-1, DOS-5). A cluster composed of muscle transcripts only present in human and mouse data is shown in blue (HOS-3, MOS-2). (B) Blow up the conserved Cell Cycle clusters (HOS-4, MOS-4, and DOS-3) showing the location of representative genes. (C) Blow up of the Immune-1 (HOS-1, MOS-4, DOS-4) and Immune-2 (HOS-8, MOS-1, and DOS-5) regions showing the location of representative genes. (D) Venn diagrams showing the number of overlapping genes observed to be commonly present in both datasets. Fishers Exact Test results indicated that the observed overlap is highly unlikely to occur by random chance. Highly significant FET (p<10⁻¹⁰) are marked with ***.

FIG. 10. Gene Cluster Expression Summary Scores (GCESS) represent the relative amount of transcript present for each cluster of genes and identify correlation between high cell cycle or low immune cell GCESSs and poor survival. (A) Overview of analyses method. (i) The log₂-transformed and mean-centered values for each gene in a cluster are summed to generate a single score (GCESS value) for each sample. Samples with high relative expression levels have large positive GCESSs while samples with low relative levels have large negative GCESSs. (ii) The GCESS scores can be used to separate the tumors into groups based on GCESS score Quartiles. (iii) The GCESS Quartile based groups can then be examined for associations with outcome using Kaplan Meier Analyses. GCESSs for human (tumors, normal bone, OS cell lines), mouse (tumors, OS cell lines), and dog (tumors, osteoblasts, OS cell lines) samples were calculated and Violin Plots were generated for (B) the “cell cycle” cluster, (C) the “immune-1” cluster, and (D) the “immune-2” cluster. Samples were ranked by GCESS and divided into quartiles groups (Q1=lowest GCESSs, Q4=highest GCESSs). KM analyses were performed, using human (n=35) and dog (n=19) samples for which survival data was known, to determine correlations between low/high GCESSs and survival. There were 17 (out of 35) death events in the human data (H052), and 19 (out of 19) death events in the dog data. (B) A significant association with shorter time to death was observed with a high cell cycle GCESS in the dog cohort, and a strong trend was also present in the human data. (C) In the human data, low Immune-1 GCESS was significantly associated with a worse survival and (D) a strong trend was present between low Immune-2 and worse survival

FIG. 11. Replication of association between high cell cycle GCESS or low immune GCESS and poor survival outcomes using array data from independent cohort. Following a similar strategy to the one used for the RNA-Seq data, 14 strong and highly correlated clusters were identified in the (A) GSE21257 dataset. Gene cluster overlap analyses comparing clusters derived from human array and human RNA-Seq data sets identified four clusters that corresponded to the conserved RNA-Seq clusters. The (B) Cell cycle cluster is labeled in red, the (C) Immune-1 cluster is labeled in green and the (D) Immune-2 cluster is labeled in purple. Fisher Exact to assess the likelihood of observing the overlap by random chance indicated that the enrichment was highly significant (p<10⁻¹⁰) for the Cell Cycle and Immune clusters. KM analyses using GCESS groups using the approach outlined in FIG. 3a showed that high levels of B) Cell cycle transcripts were associated with worse outcomes and significantly increased likelihood of tumor metastasis. Low levels of (C) Immune-1 and (D) Immune-2 transcripts were associated with significantly worse survival and significantly increased likelihood of tumor metastasis (E) Metastatic Samples have lower Immune-2 transcript levels in mouse and human samples. MOS-1 GCESS values were lower in tumors from mice where metastatic lesions were observed during necropsy (p<0.05). Tumors from Human patients with metastases present at diagnoses showed a trend towards lower Immune-2 GCESS scores in both the RNA-SEQ data as well as the array data and this trend became increasingly significant in the array data when tumors where metastases were observed in the patient within one year (p<0.001) or at any point (p<0.0001) were included with the patients with metastases at diagnosis.

FIG. 12. Cell cycle, Immune-1, Immune-2, and muscle gene clusters in humans (HOS), mouse (MOS), and GSE21257 dataset. Cell cycle, Immune-1, and Immune-2 gene clusters in dogs (DOS).

### DETAILED DESCRIPTION OF ILLUSTRATIVE EMBODIMENTS

Human osteosarcoma (OS) is a rare bone disease primarily affecting children and adolescents that has proven challenging to study, due both to its rarity and its genetic heterogeneity. Osteosarcoma is very common in large breed dogs and can be genetically induced in mice, making the disease ideal for comparative genomic analyses across species. The overall survival rate of patients with metastatic osteosarcoma has not improved over the past three decades. Frequently, current standard-of-care therapies ultimately fail to prevent relapse and metastasis. Understanding how tumor heterogeneity is conserved across species and associated with progression to metastasis resulting in poor outcomes will enable more efficient development of effective strategies to manage osteosarcoma and improve therapy.

This disclosure describes the use of transcriptional profiles of osteosarcoma tumors and cell lines derived from humans, mice, and dogs. Conserved heterogeneity was present in tumors from all three species. The conserved heterogeneity included transcript clusters associated with cell cycle/mitosis and with infiltrating immune cells. Further, this disclosure describes a novel Gene Cluster Expression Summary Score (GCESS) technique for quantifying tumor heterogeneity. The GCESS values are associated with patient outcome. Specifically, human osteosarcoma tumors with GCESS values indicative of decreased immune cell presence are associated with metastasis and poor survival. These results were validated in an independent human osteosarcoma cohort and in 15 different tumor data sets obtained from The Cancer Genome Atlas (TCGA, http://tcga-data.nci.nih.gov/tcga).

In humans, osteosarcoma is frequently observed in adolescents with a second incidence peak in adults >60 years old. The 5-year survival rate for patients with metastatic osteosarcoma is only 15-30%, representing a significant loss-of-life impact for these pediatric patients. In elderly patients, osteosarcoma is often the result of a transformation of a benign bone lesion, previous exposure to radiation, or Paget's disease. In all age groups, a common primary osteosarcoma tumor site is in the lower long bones. Osteosarcoma in dogs is much more common, with an overall incidence rate 30-50 times higher than humans, and a lifetime risk up to 20% in larger, pre-disposed breeds. Human and dog osteosarcoma share many clinical and molecular features and insight gained from one species may be translatable to the other. Osteosarcoma tumors can also be generated in wildtype and predisposed mice via tissue specific mobilization of the T2/ONC transposon by the Sleeping Beauty Transposase.

Osteosarcoma is characterized by complex karyotypes with highly variable structural and numerical chromosomal aberrations in humans, dogs, and mice. Sequencing studies have identified recurring genetic alterations in osteosarcoma, some of which are common to both human and dog osteosarcomas. However, these genetic alterations have not yet been translated into new clinical treatment strategies and improved outcomes for osteosarcoma patients. There remains a critical need to identify ways to identify risk of osteosarcoma progression at an early, pre-metastatic stage in both human and dog patients.

This disclosure describes an approach for integrating transcriptional data from multiple species by identifying large gene sets with expression patterns that are conserved across human, dog, and mouse osteosarcoma cohorts. Through this approach, conserved gene signatures were identified. The first signature included genes involved in cell division and DNA damage response. Additional signatures were enriched for genes expressed by immune cells. As briefly summarized above, this disclosure also describes a novel Gene Cluster Expression Summary Score (GCESS) technique for quantifying heterogeneity. The GCESS technique allows one to evaluate survival outcomes and/or metastasis in osteosarcoma patients. The GCESS technique allows for dimensional reduction of gene expression data and minimizes the potential for false-positive results derived from multiple testing. Higher cell cycle GCESSs and lower immune cell GCESSs were significantly associated with worse patient outcomes and metastasis.

Osteosarcoma Tumors Show a Common Transcriptional Profile Across Species that is Distinct from Other Types of Tumors

To better understand the osteosarcoma transcriptome, RNA-Seq analysis was performed on mRNA libraries generated from human, dog, and mouse tumors and cell lines (Table 1). While described below in the context of an exemplary embodiment in which the transcriptome was analyzed using RNA-Seq, the method may be performed using any technique that allows one to measure transcription or protein expression such as, for example, multiplexed quantitative real Time PCR(qRT-PCR), quantitative nuclease protection assay (qNPA), etc.

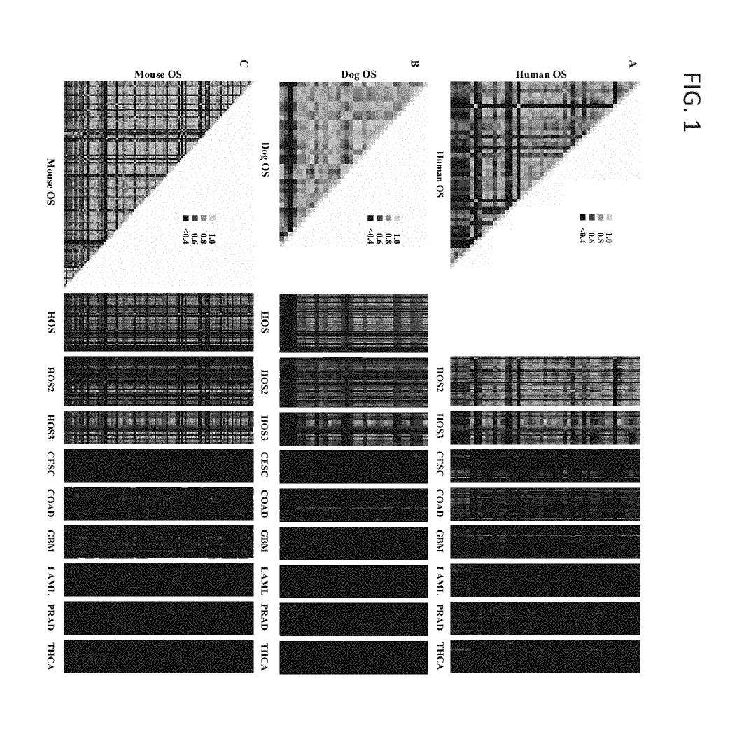

Despite the highly complex karyotypes associated with osteosarcoma, tumors maintained common transcriptional characteristics between species that were distinct from other types of tumors. This was established by calculating pairwise Pearson correlation coefficients within and between samples, revealing strong correlations between tumors within and across each of the three species cohorts (FIG. 1). Strong correlations were observed within each human cohort (average intra-cohort correlations: HOS1 0.62, HOS2 0.67, HOS3 0.66). Strong correlations also were observed between the human cohort (HOS) and two independent human osteosarcoma tumor cohorts (HOS2, HOS3) recently published by other groups (Chen et al., 2014. Cell Reports 7(1):104-112; Perry et al., 2014. Proceedings of the National Academy of Sciences USA 111(51):E5564-E5573).

TABLE 1
RNA-Seq samples used in this stiudy

| | | | Cell | | |
|---|---|---|---|---|---|
| Source | Species | OS tumors | lines | Other | Total |
| this study | Human | 44 | 5 | 3 normal bone | 52 |
| this study | Mouse | 92 | 11 | | 103 |
| this study | Dog | 31 | 2 | 1 osteoblast | 34 |
| | | | | cell line | |
| Perry et al.¹ | Human | 35 | | | 35 |
| Previous Studies^{2,3,4} | Human | 25 | | | 25 |

¹Proceedings of the National Academy of Sciences of the United States of America. 2014; 111(51): E5564-5573.
²Moriarity et al., 2015. Nat Genet 47(6): 615-625.
³Man et al., 2004. BMC Cancer 4: 45.
⁴Chen et al., 2014. Cell Reports 7(1): 104-112.

Despite the established genetic heterogeneity characteristic of osteosarcoma, the three HOS cohorts were much more similar to each other than to any other examined human tumor type (e.g., cervical, colorectal, glioblastoma, leukemia, prostate adenocarcinoma, and thyroid cancer) obtained from TCGA, indicating that there are common transcriptional events that define osteosarcoma pathology (FIG. 1A).

To establish the cross-species relevance of the dog (DOS, FIG. 1B) and mouse (MOS, FIG. 1C) osteosarcoma tumors, both interspecies and intraspecies correlations were calculated. Similar to humans, intraspecies correlations were high in both dog (0.76) and mouse (0.68) samples. Dog and mouse osteosarcoma samples were both highly correlated with the human osteosarcoma tumors (HOS1-DOS 0.48, HOS1-MOS 0.52), but not with other human cancers (DOS-cervical 0.18, MOS-cervical 0.14, DOS-colorectal 0.31, MOS-colorectal 0.28, DOS-glioblastoma 0.21, MOS-glioblastoma 0.25, DOS-leukemia 0.14, MOS-leukemia 0.14, DOS-prostate adenocarcinoma 0.24, MOS-prostate adenocarcinoma 0.21, DOS-thyroid cancer 0.13, MOS-thyroid cancer 0.12), validating a cross-species analysis strategy to identify common transcriptional components in the development and progression of osteosarcoma.

### OS Tumors Show Common Transcriptional Heterogeneity Across Three Species

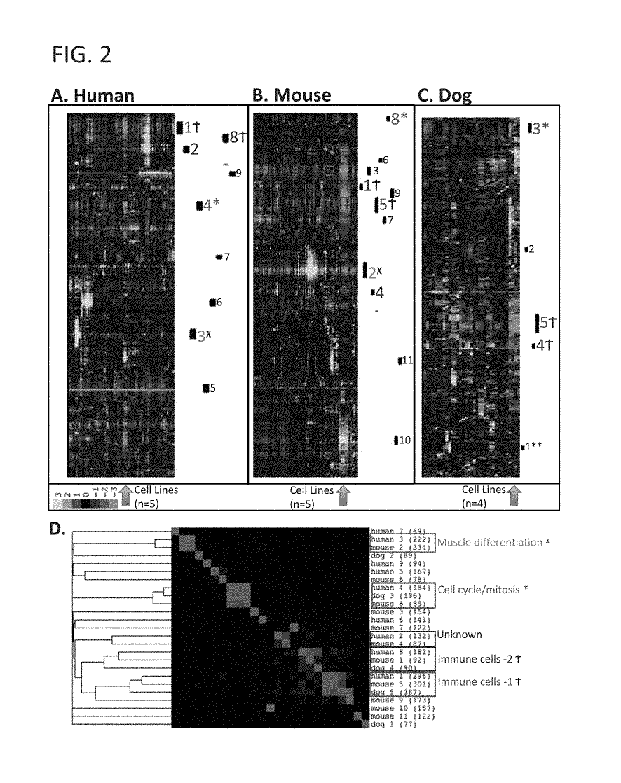

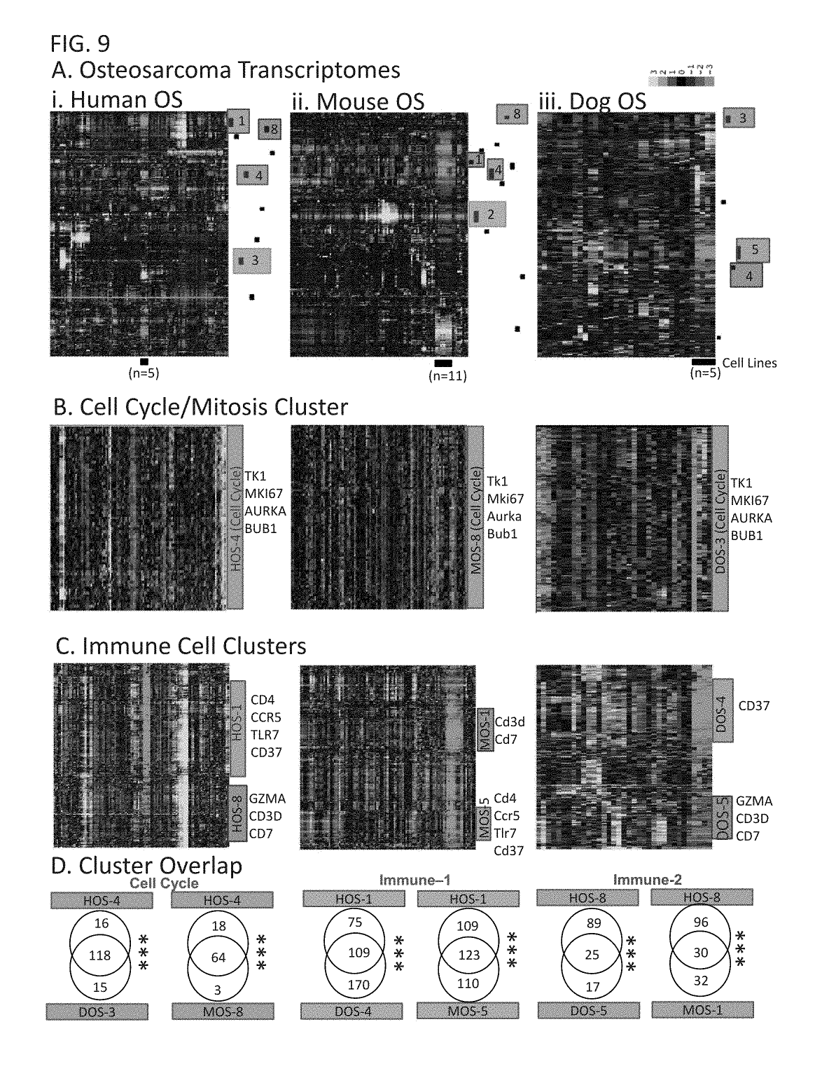

To systematically assess transcriptional heterogeneity across human, dog, and mouse osteosarcoma tumors each dataset was first analyzed independently using average linkage clustering. Tightly correlated and large gene clusters were defined as having both a dendrogram node correlation greater than 0.6 and a minimum of 60 genes. A total of nine human, five dog, and 11 mouse gene clusters were identified (FIG. 2A-C; FIG. 9A). To ensure that these clusters were not artifacts, similarly sized random datasets and shuffled permutations of the real data were generated and clustered. No clusters passed the threshold criteria in either the random or permuted datasets, validating that the clusters observed in the real RNA-Seq data represent true transcriptional heterogeneity.

To determine whether the identified gene clusters represented sets of genes common across the three species, pairwise percent overlaps were calculated between each gene cluster from a single species and all clusters in the other two species. The resulting percentage overlap values were clustered for each species pair. Several clusters of genes showed clear overlap across species (FIG. 2D; FIG. 9B-E), indicating conserved patterns of transcriptional heterogeneity in osteosarcoma tumor samples.

To describe the potential biological significance of these common gene clusters, enriched functional annotations were identified using IPA software (Qiagen). The most conserved cluster across species was composed of transcripts that were independently highly enriched in genes associated with cell cycle and mitotic functions. The next two highly conserved clusters across species were each independently enriched in transcripts relating to immune cell functions. A gene cluster enriched for muscle differentiation genes was observed in the human and mouse tumors (FIG. 2D) but it was not present in the dog tumors (FIG. 2D; FIG. 9D).

Genes in the two immune cell gene clusters represented those whose expression is increased in particular leukocytes. The immune cell gene clusters were compared to a gene signature matrix (LM22) that consists of 547 genes and was created to identify genes unique to 11 different subtype populations of immune cells (Newman et al., 2015. Nature Methods 12(5):453-457). Overall, the human immune cell annotated gene clusters were highly enriched in the LM22 gene transcripts. Using the LM22 annotations to determine the types of active leukocytes present (as represented by their unique transcripts), genes representing monocytes to be most enriched in human cluster-1 (immune cells-1 as defined in FIG. 2) and genes representing T cells to be most enriched in human gene cluster-8 (immune cells-2 as defined in FIG. 2) were assessed. Results from assessing enrichment of the LM22 genes in the dog and mouse immune cell gene clusters also supported these results.

### The Gene Cluster Expression Summary Score (GCESS) Technique Quantifies Tumor Heterogeneity

The Gene Cluster Expression Summary Score (GCESS) was developed to generate a normalized and easily comparable number for use in further association analyses. The GCESS is defined as the sum of expression values—log₂-transformed and mean centered—of all genes in a particular gene cluster for a single sample. A negative GCESS indicates relative underexpression of the group of genes in that sample compared to all of the samples in the analysis set, a positive GCESS indicates overexpression, and a GCESS close to 0 (zero) indicates mean expression. The magnitude of the GCESS value depends on both the number of genes present in the cluster and the extent of transcriptional variation observed across samples.

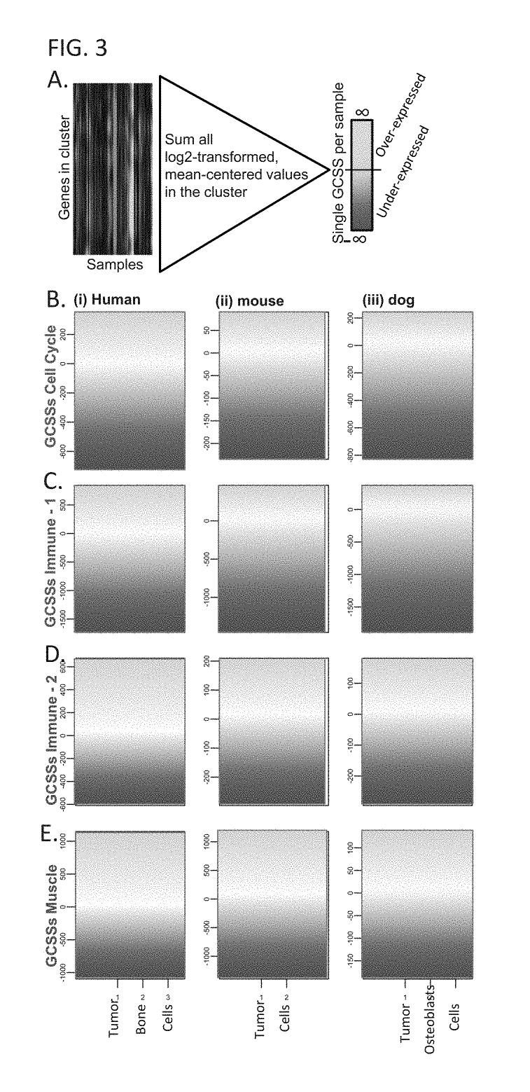

To better characterize the conserved osteosarcoma tumor heterogeneity, GCESS values for the identified cell cycle/mitosis gene cluster, immune cell gene cluster, and muscle differentiation gene cluster in each sample were calculated. In addition to human, dog, and mouse osteosarcoma tumor samples, tumor-derived cell lines, normal human bone cells, and dog osteoblasts also were analyzed. The distribution of GCESS values (FIG. 3) shows distinct patterns for cells/cell lines, normal bone, and tumor samples.

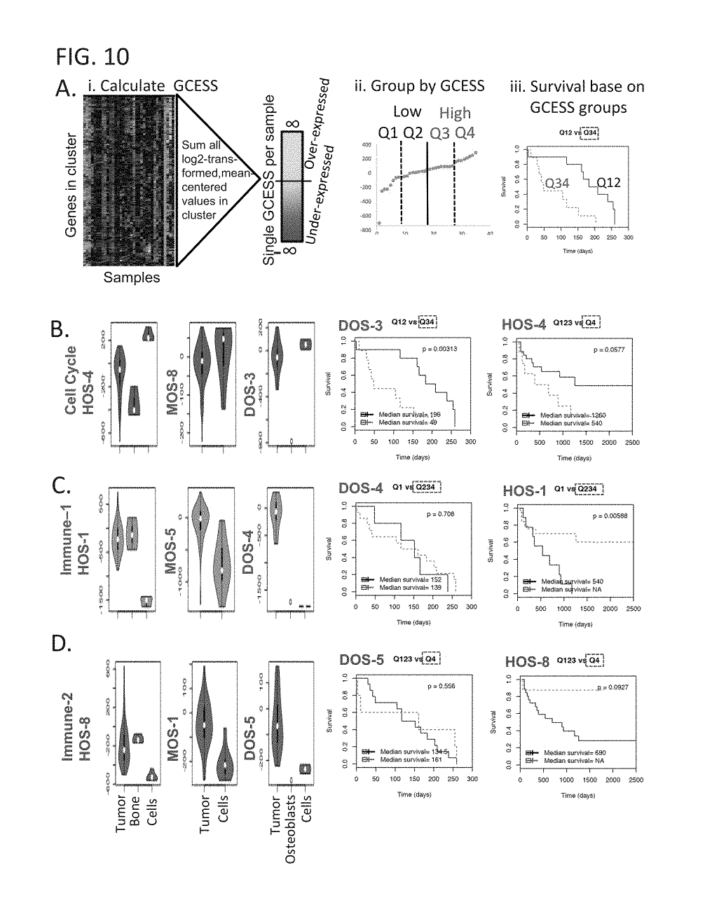

Tumor-derived cell lines had higher cell cycle GCESS values compared to tumor tissues, while normal human bone had cell cycle GCESS values corresponding to the lower range of GCESS values obtained from tumor samples (FIG. 3B; FIG. 10B). Dog osteoblasts had an extremely low cell cycle GCESS. These results are consistent with the upregulation of cell cycle genes in a subset of highly proliferating osteosarcoma tumors.

Tumor-derived cell lines had lower immune GCESS values compared to most tumor tissues in all three species datasets. The immune cell GCESS of the normal human bone sample was within the middle of the range seen in human tumor samples. Dog osteoblasts had GCESS values corresponding to the tumor-derived cell lines (FIG. 10C-D). These results are consistent with the absence of immune cells in cell lines and the variable presence of immune cells in tumor-derived tissues from all three species.

The expression variability in immune cell genes may be due, at least in part, to differing quantities of infiltrating and/or proliferating immune cells in the tumor samples. The cell lines, which should represent pure populations of malignant osteoblastic cells, had the lowest immune GCESS values. The immune cell GCESS of the normal human bone sample was at the high end of the range seen in human tumor samples (FIG. 3C-D).

The muscle differentiation GCESS values also were highly variable in human and mouse tumors (FIG. 3E). A muscle differentiation cluster was not identified in the dog data based on variable expression (FIG. 2). Therefore, the genes of the human muscle differentiation cluster (human cluster-3) were used as a proxy gene set for calculating dog gene cluster GCESS values. These genes showed very low variability in the dog dataset and were expressed at very low levels, indicating that active muscle differentiation was not present in the dog osteosarcoma cohort.

To validate the variable presence of immune cells indicated by GCESS immune cluster scores, 10 FFPE sections derived from canine OS tumors were evaluated and sequenced for the presence of T cells and macrophages within tumor stroma by immunohistochemistry. Immunohistochemistry staining supported the transcriptome-derived GCESS data for both MAC387 and CD3 staining. Stromal MAC387 was not observed in the three tumors with the lowest GCESS scores for the immune cell cluster-1 and occasional MAC387 positive cells were observed for the three tumors with the highest immune cluster-1 GCESS scores. One of the middle range tumors showed the presence of MAC387 positive staining cells within the stroma while three others did not. For CD3, only the sample with a positive GCESS immune cluster-2 score showed T-cells present within the tumor stroma. Of note, the next four tumors sorted by immune cluster-2 GCESS score all showed MAC387 positive cells within the tumor stroma.

### Minimization of Multiple Testing Errors

Associations between osteosarcoma patient outcomes and individual gene transcript levels are commonly calculated using the Kaplan-Meier (KM) estimator, a non-parametric statistic that estimates the survival function based on censored lifetime data. KM survival analyses were calculated using sample groups defined by individual transcript levels for the dog and the human HOS2 datasets. Potentially significant events were present following comparison of all transcripts (p<10⁻⁵) used in clustering. To ensure that these associations were not false positives, similarly sized random datasets and shuffled permutations of the real data were also analyzed. Comparing results obtained from random data versus real data revealed that random associations led to the same level of significance as the real data, indicating that methodological improvements were necessary.

Two routes are typically used to minimize the effects of multiple testing in statistical analyses. The first entails increasing sample numbers to surpass multiple testing corrections via increased statistical power. This was not feasible in this case. A second approach, which was used here, was to drastically decrease the number of tests performed by testing the gene cluster GCESS values rather than all individual transcript values. The GCESS was used to rank the tumors into quartile (Q) groups and systematically compare Q1-(lowest GCESSs) vs-Q234, Q12-vs-Q34, and Q123-vs-Q4. (FIG. 10A). Using this approach, associations between outcome and immune cell GCESS could be systematically examined without prior knowledge of the type of association present. Examination of randomly generated and real, but permuted datasets using the full pipeline, did not identify gene clusters, limiting association analyses to gene clusters with strong support.

Systematic Examination of GCESSs with Tumor Outcomes Identifies Poor Patient Outcome Association with High Expression of Cell Cycle Cluster Genes and Low Expression of Immune Cell Cluster Genes

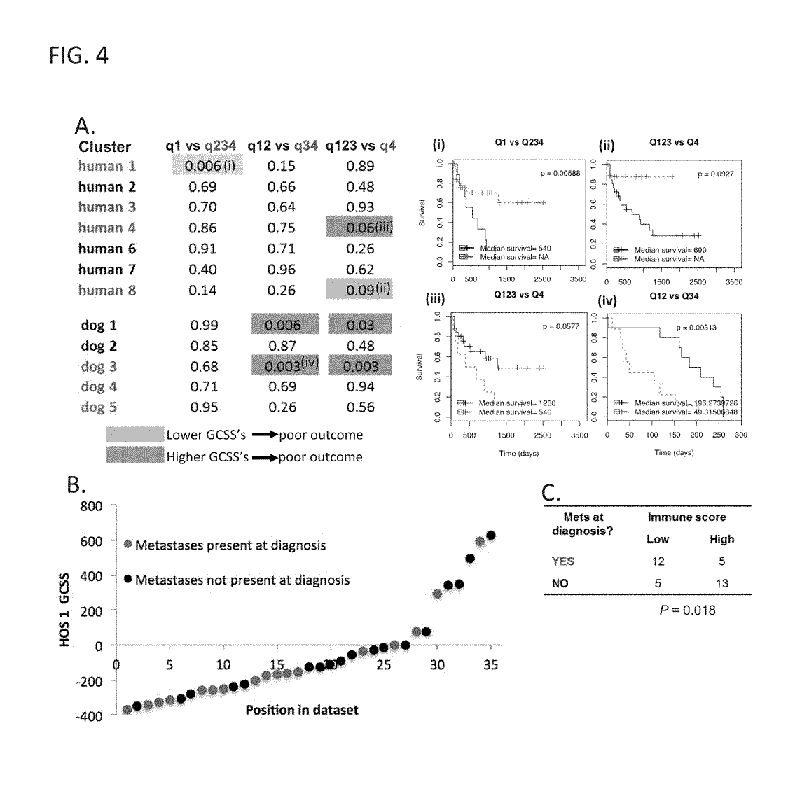

Applying the GCESS approach to the dog data revealed an association between increased transcript levels of cell cycle genes and poor outcomes. Significantly worse outcomes were associated with the higher cell cycle GCESSs in both the Q123-vs-Q4 and Q12-vs-Q34 comparisons. Similarly, the human data analysis revealed that the highest cell cycle GCESSs (Q4) also had a strong trend towards worse outcomes (Q123-vs-Q4). Analyzing the human immune cell cluster-1 (human gene cluster-1) GCESSs demonstrated that patients with the lowest immune cell cluster GCESSs had significantly worse outcomes (Q1-vs-Q234). In addition, the human immune cell cluster-2 (human gene cluster-8) also showed a trend towards association between low GCESSs and worse outcomes (FIG. 4; FIG. 10).

Low Immune Cell GCESS Associated with Metastasis at Diagnosis

Of the 35 human osteosarcoma patients with survival outcomes in the HOS2 dataset, 17 (49%) presented with metastasis at the time of initial diagnosis. To determine whether patients whose tumors had lower immune cell GCESSs were more likely to present with metastasis, GCESSs were plotted and patients with metastasis were annotated. Patients with lower immune cell GCESSs were significantly more likely to have metastasis at diagnosis (p=0.018, Fishers exact test) (FIG. 4).

### Association Between GCESS and Patient Outcome Validated in Independent Cohort

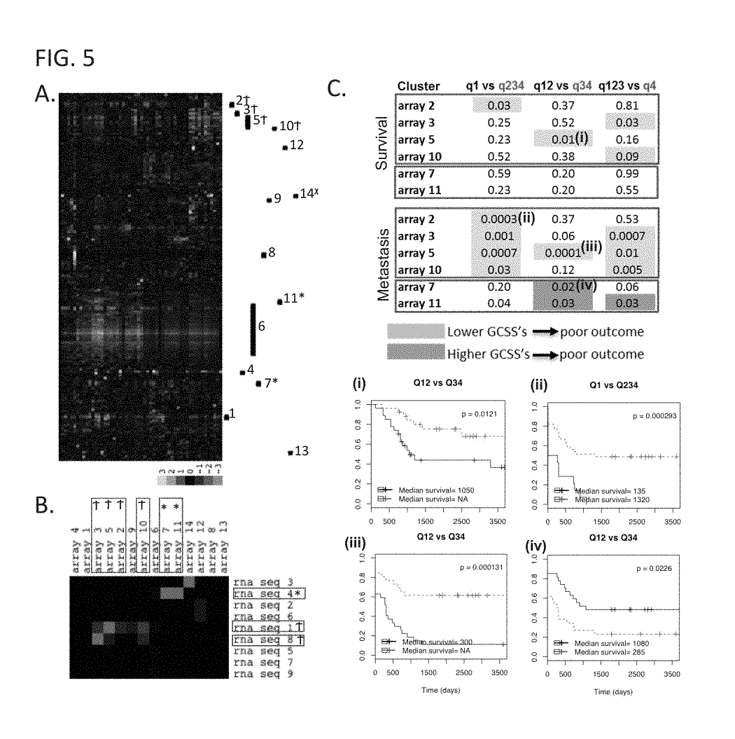

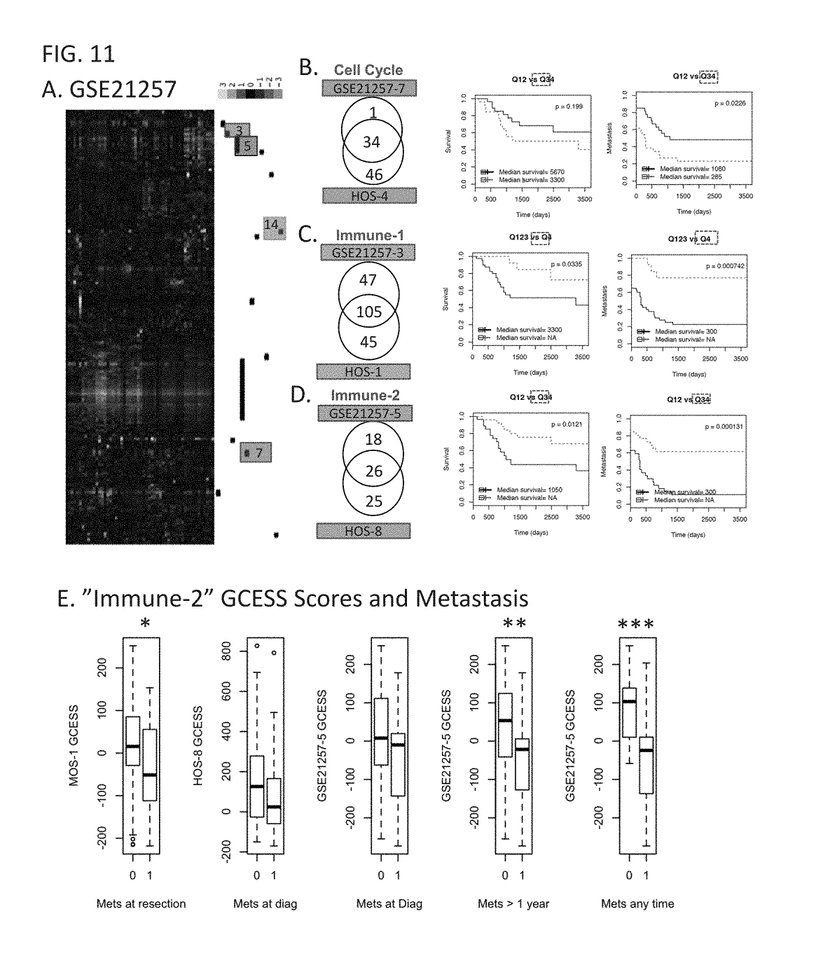

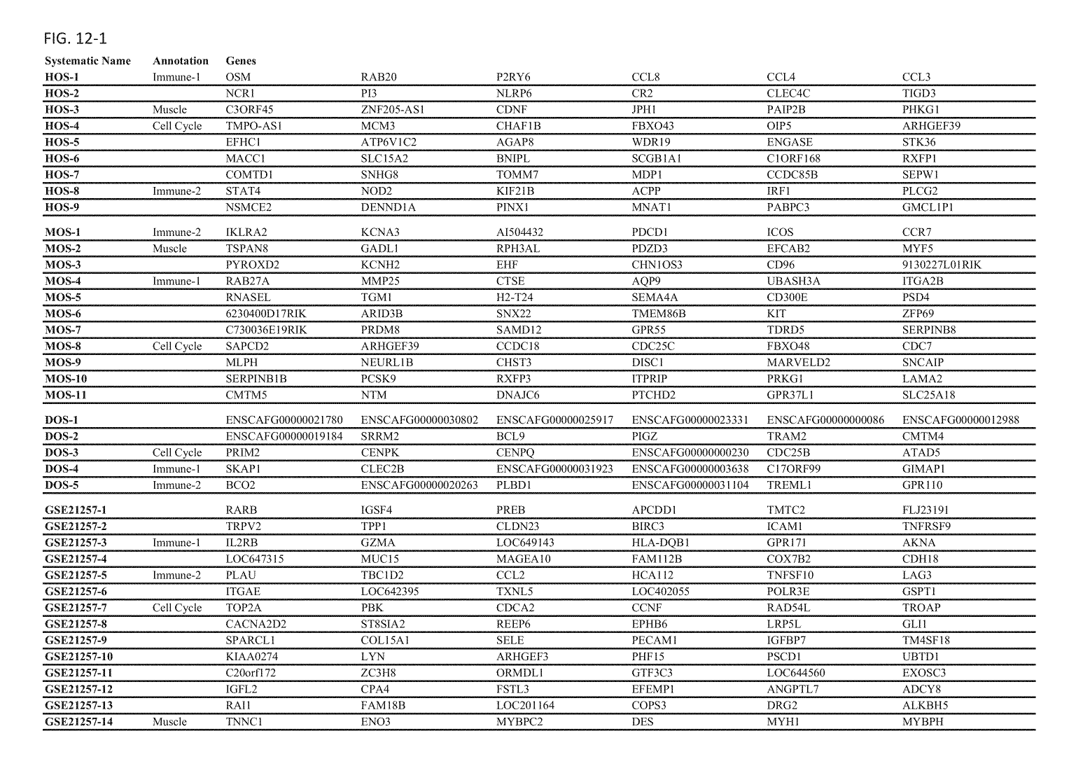

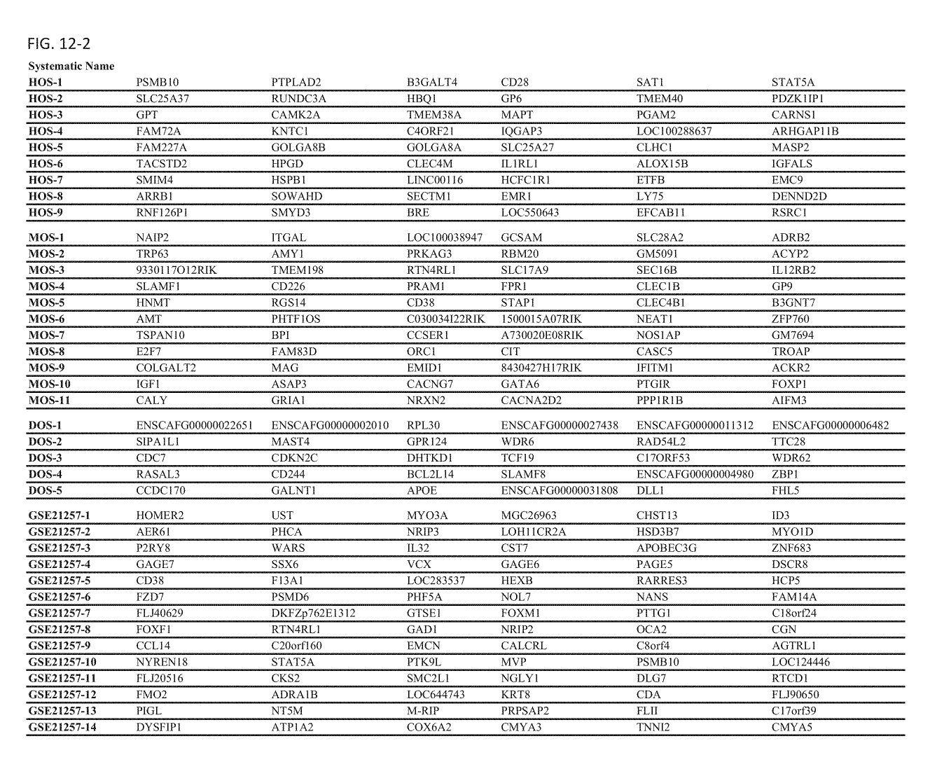

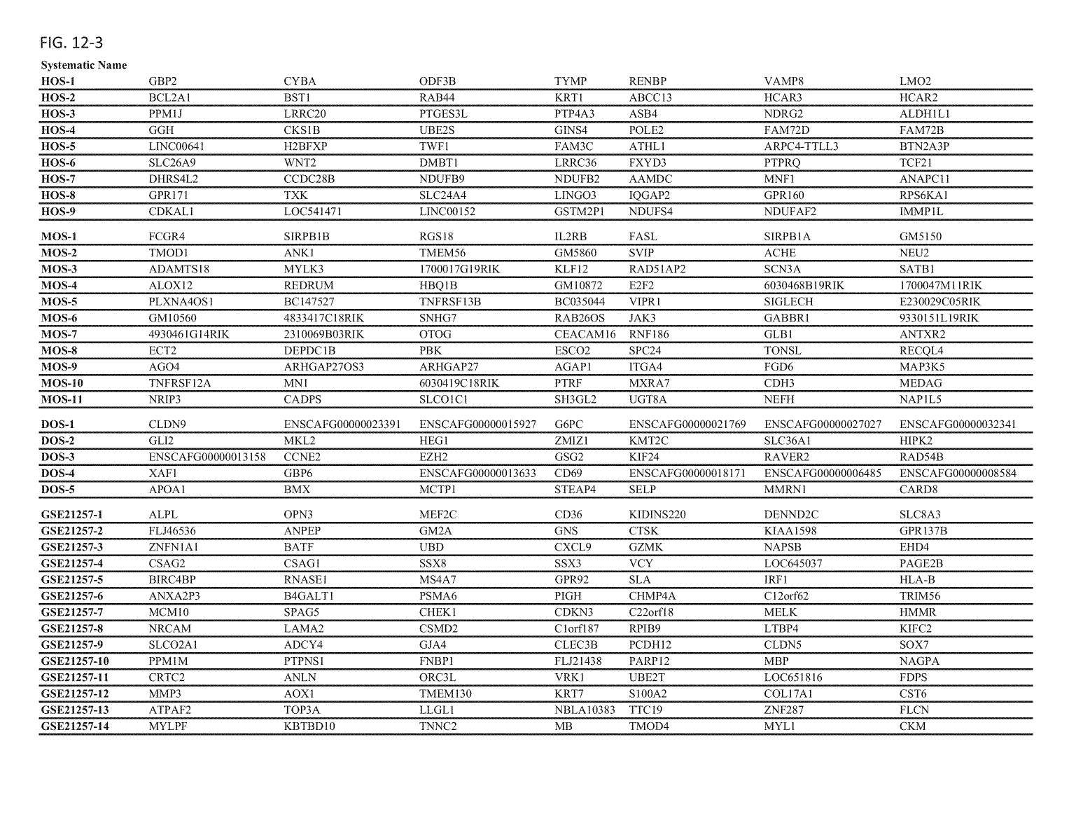

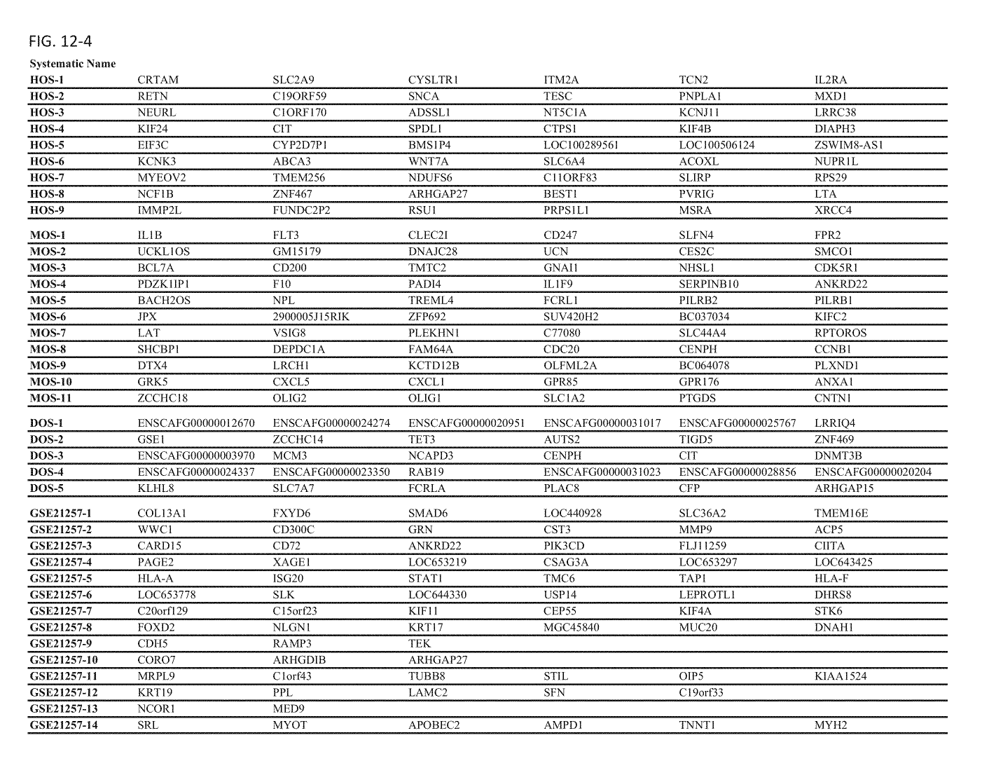

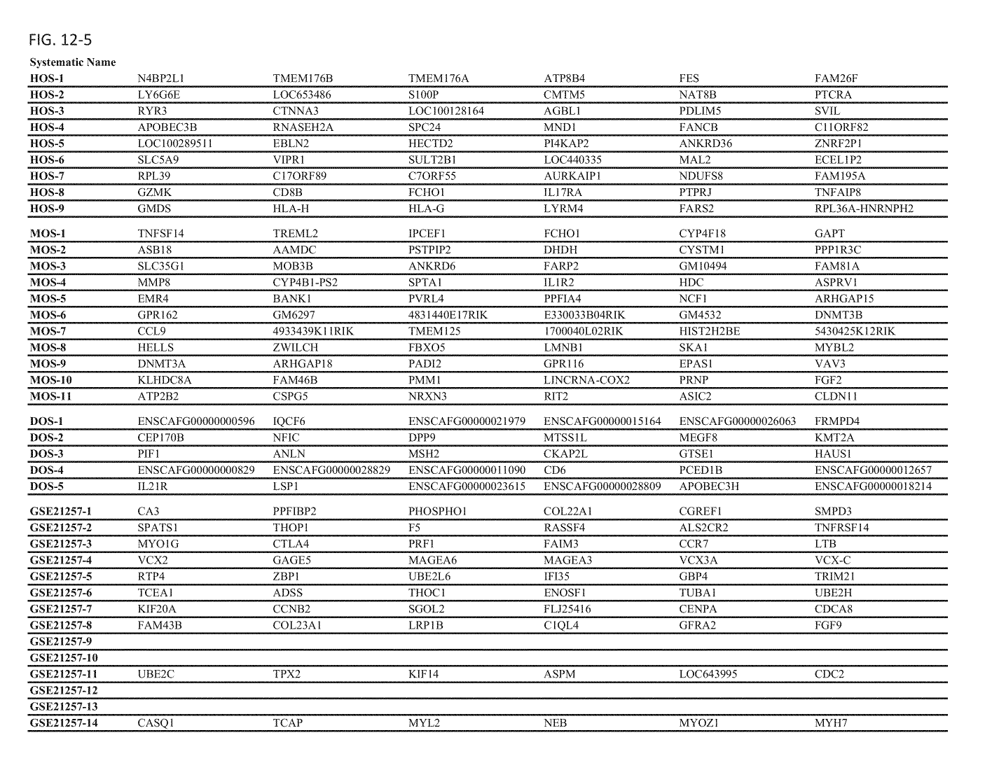

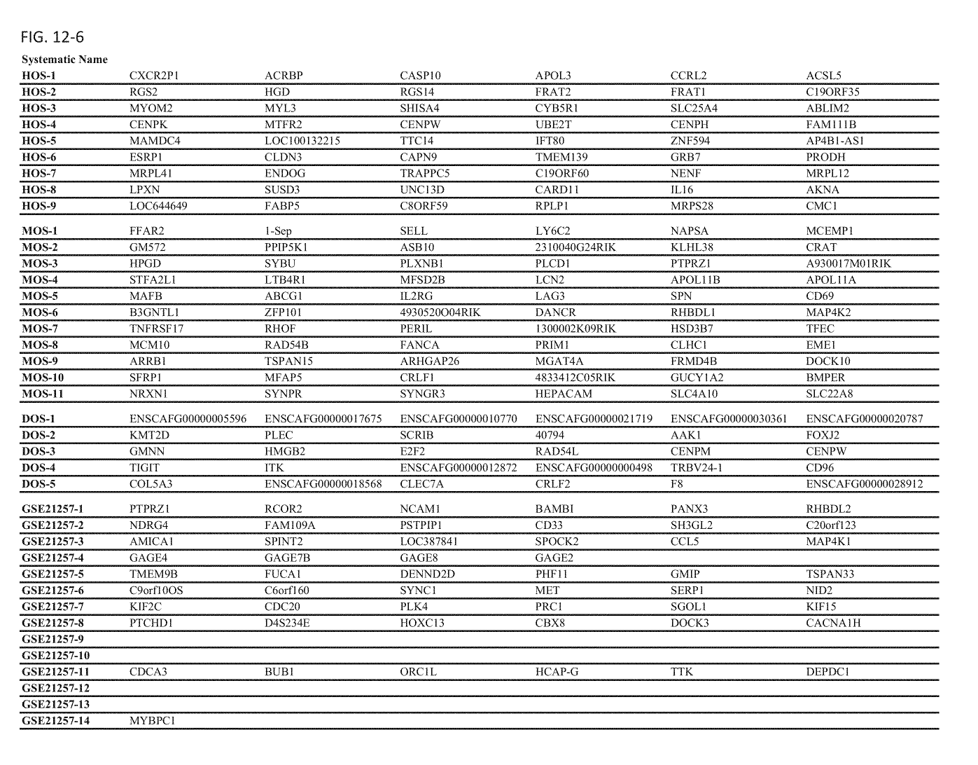

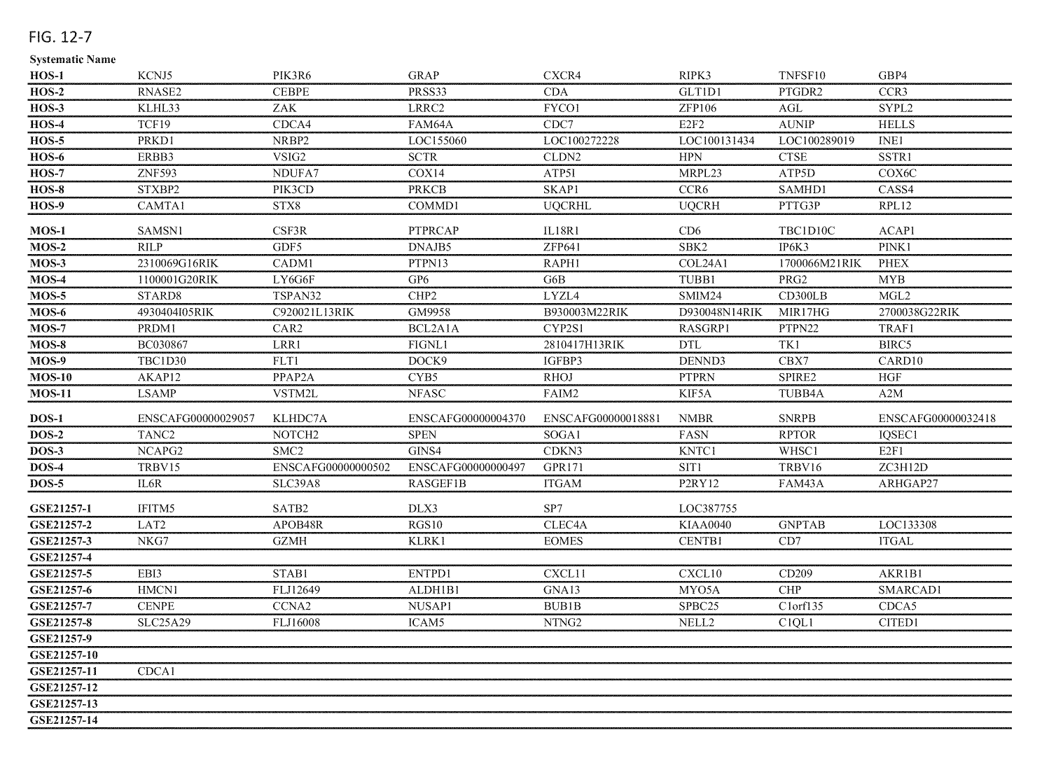

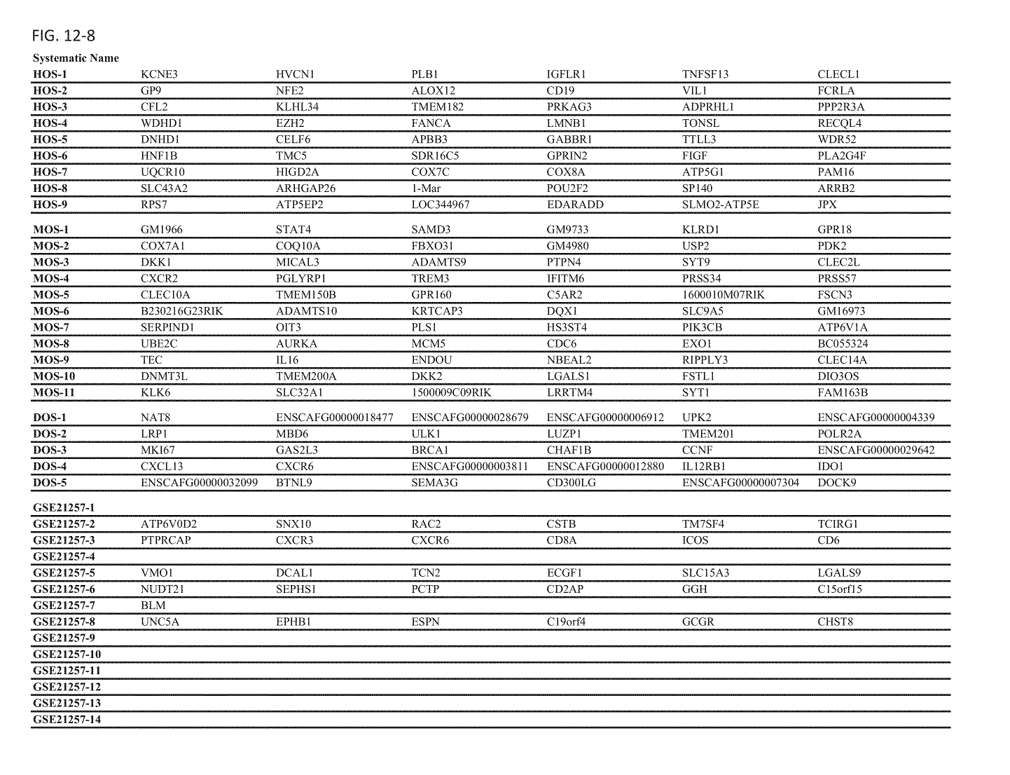

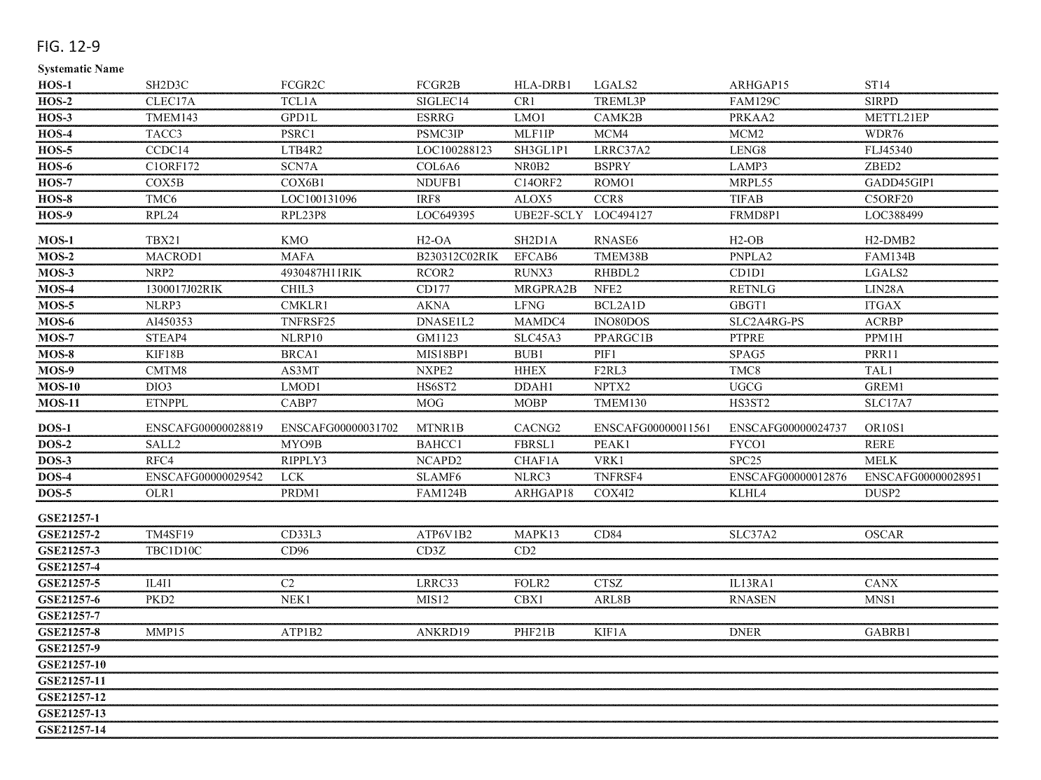

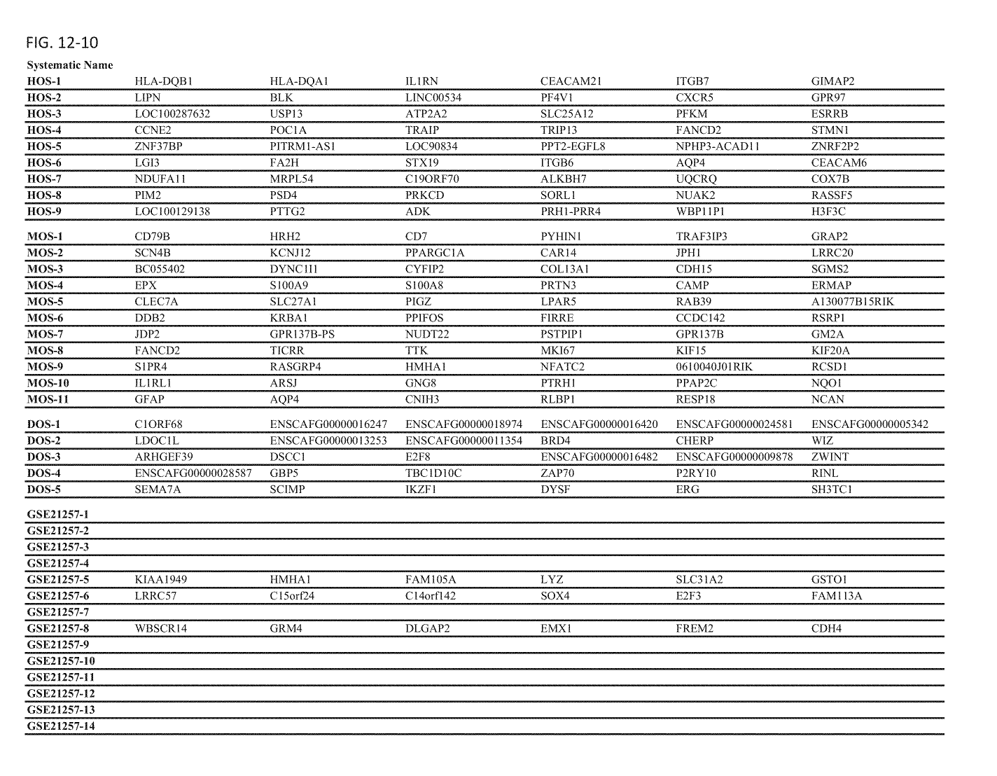

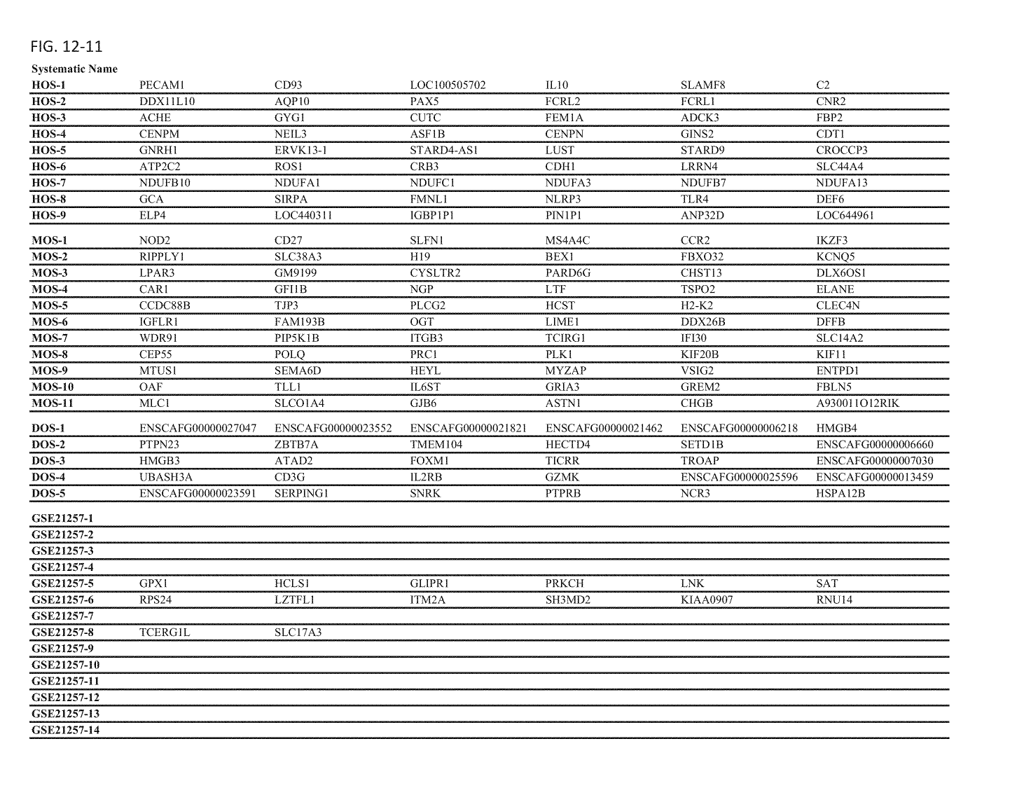

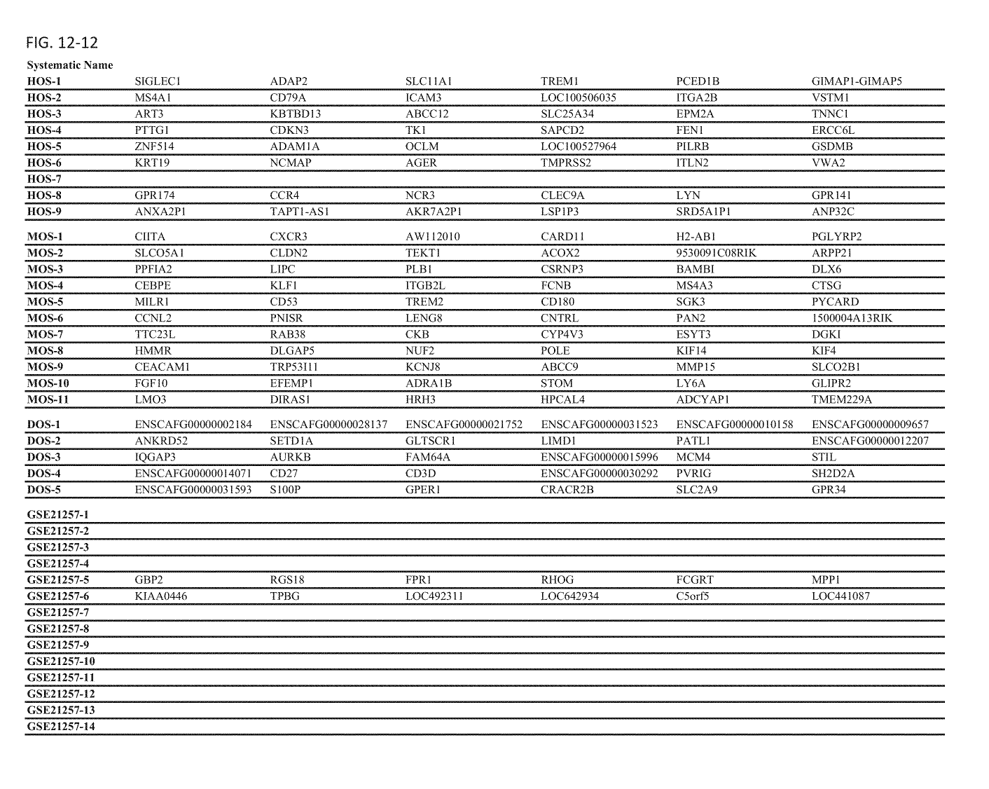

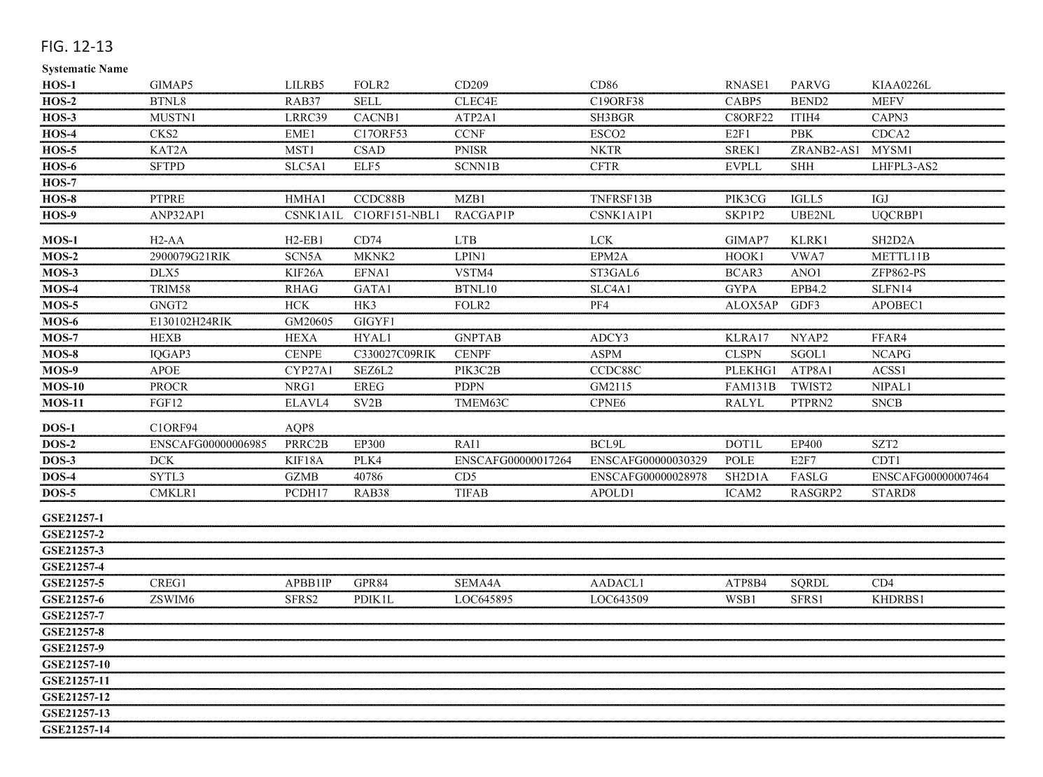

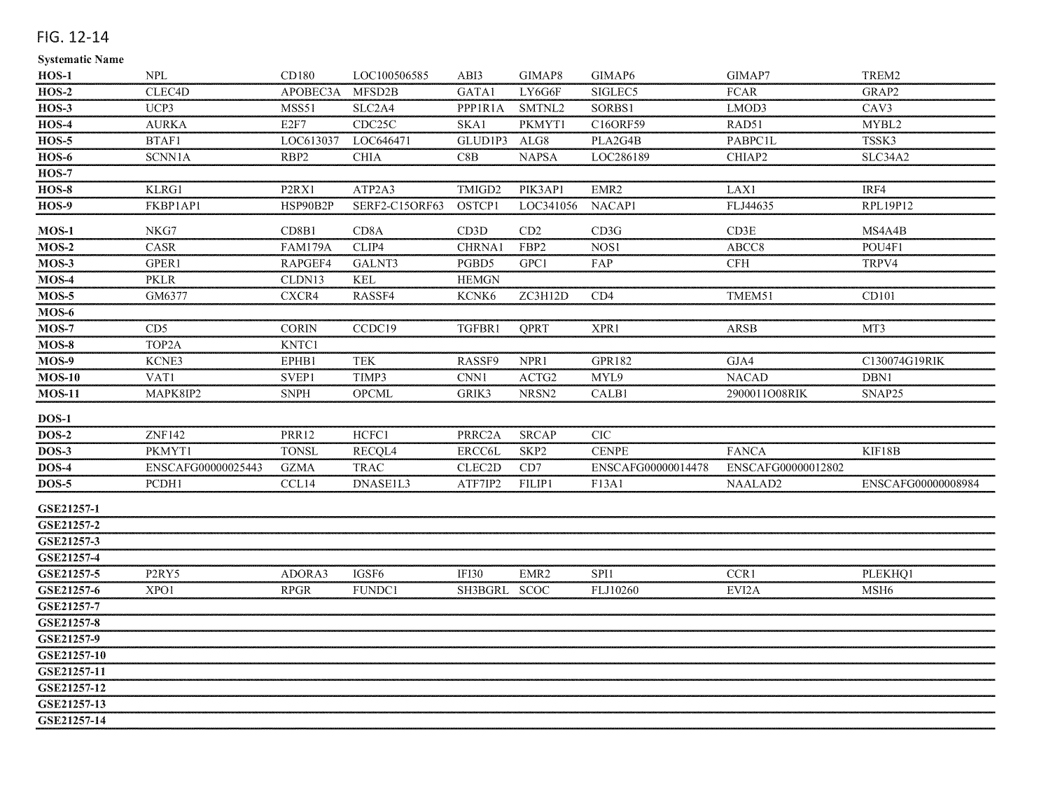

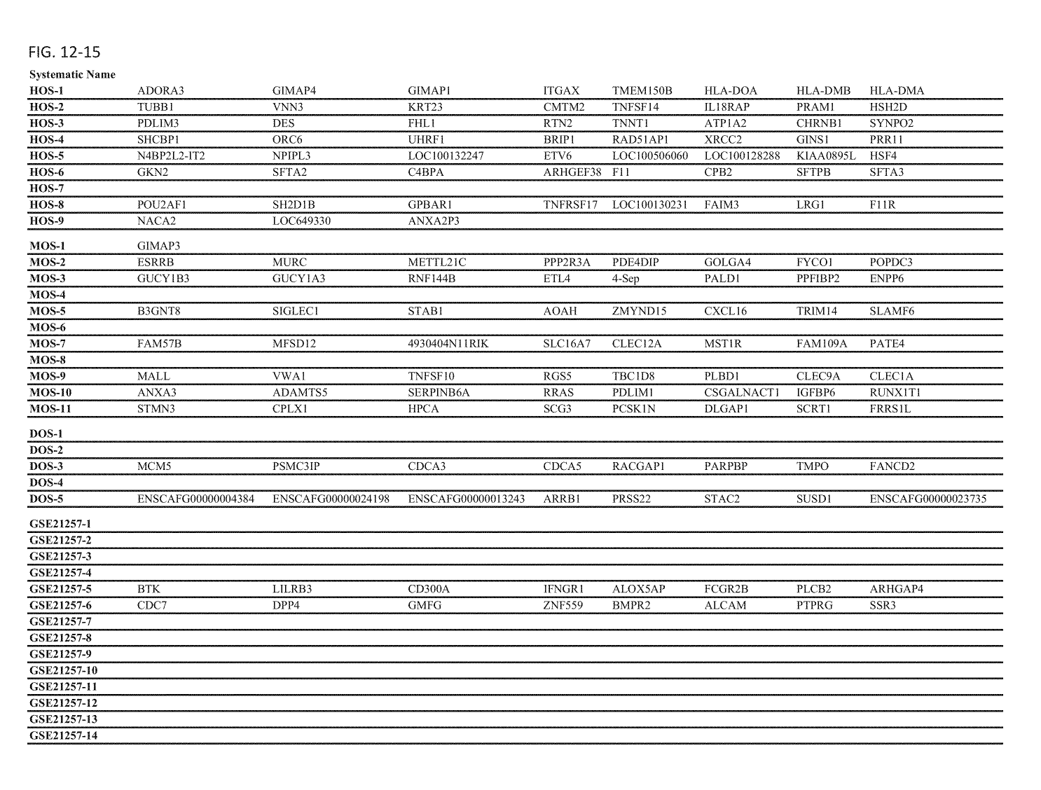

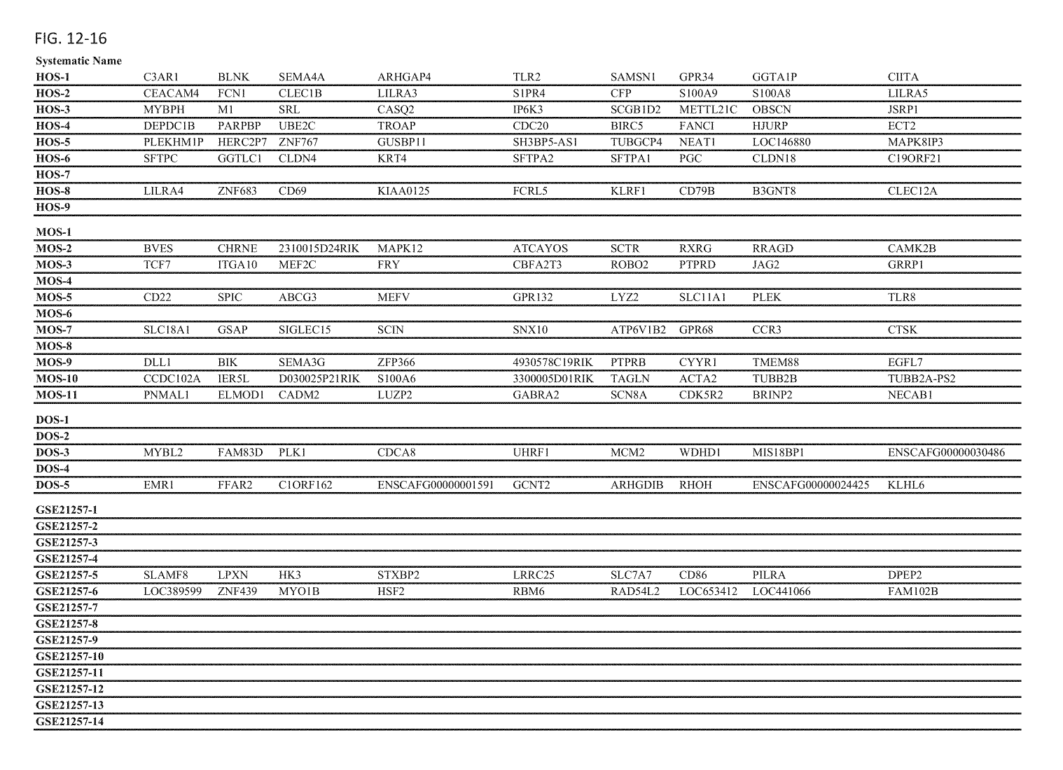

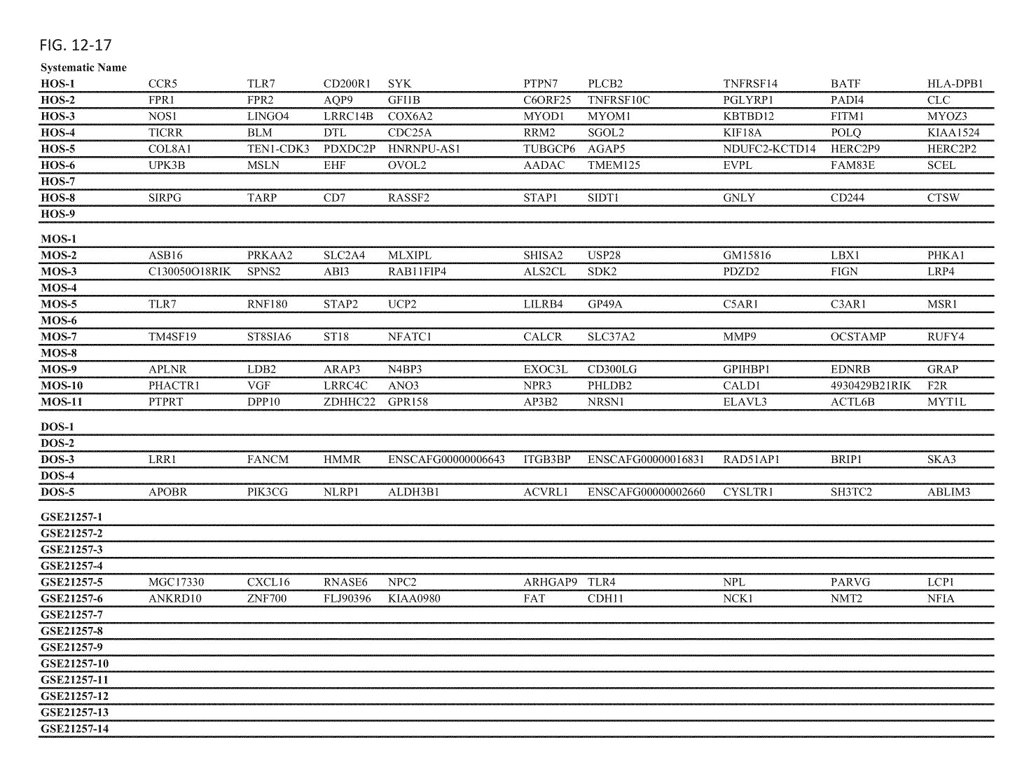

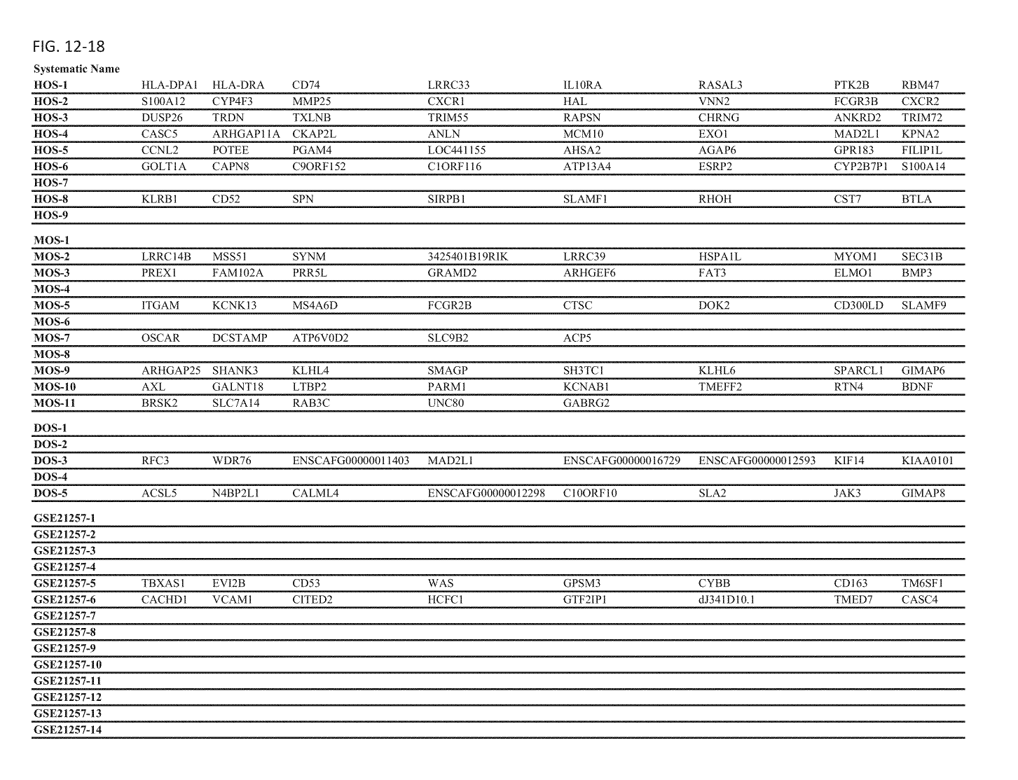

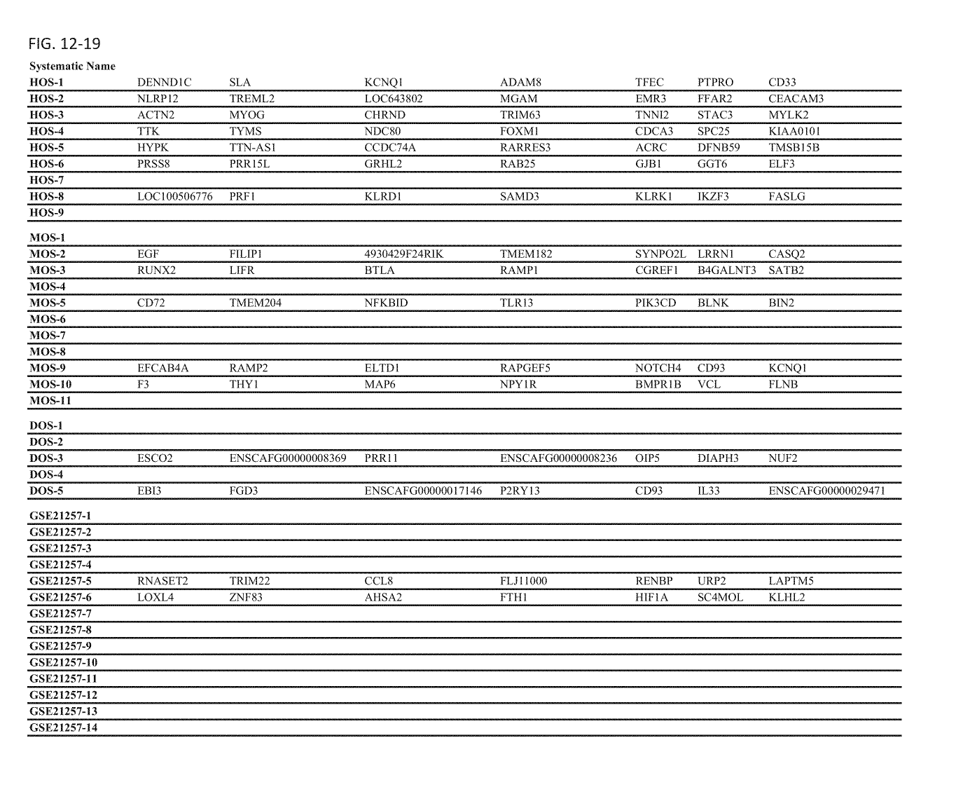

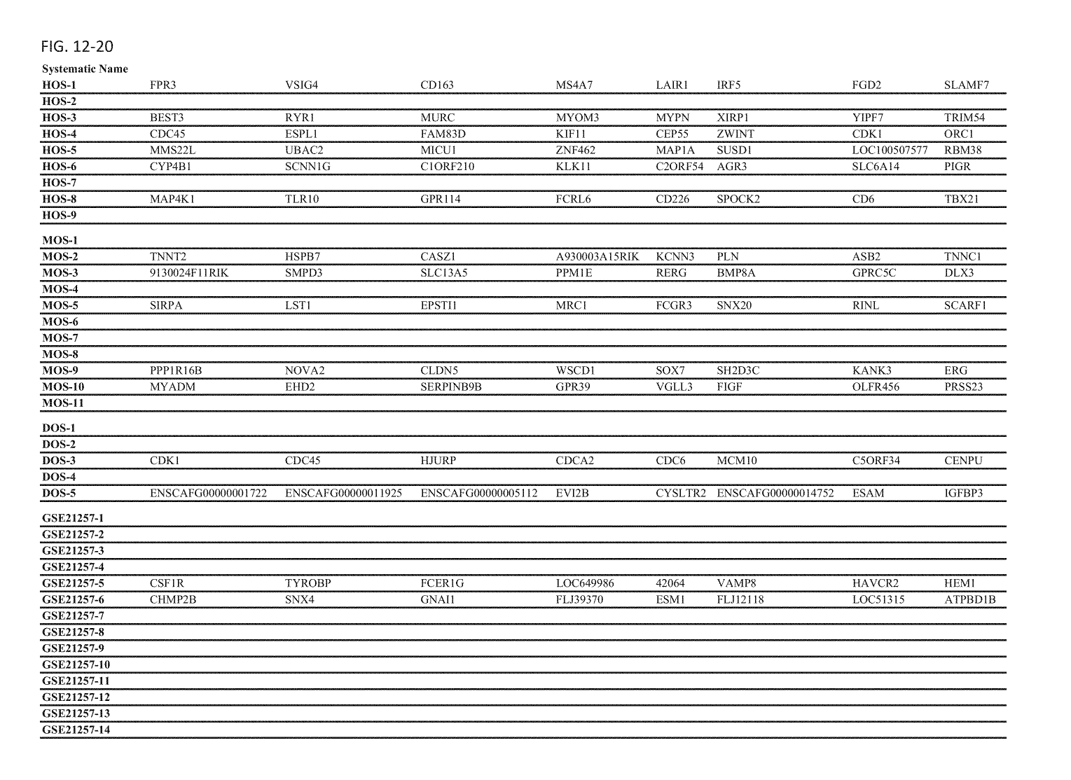

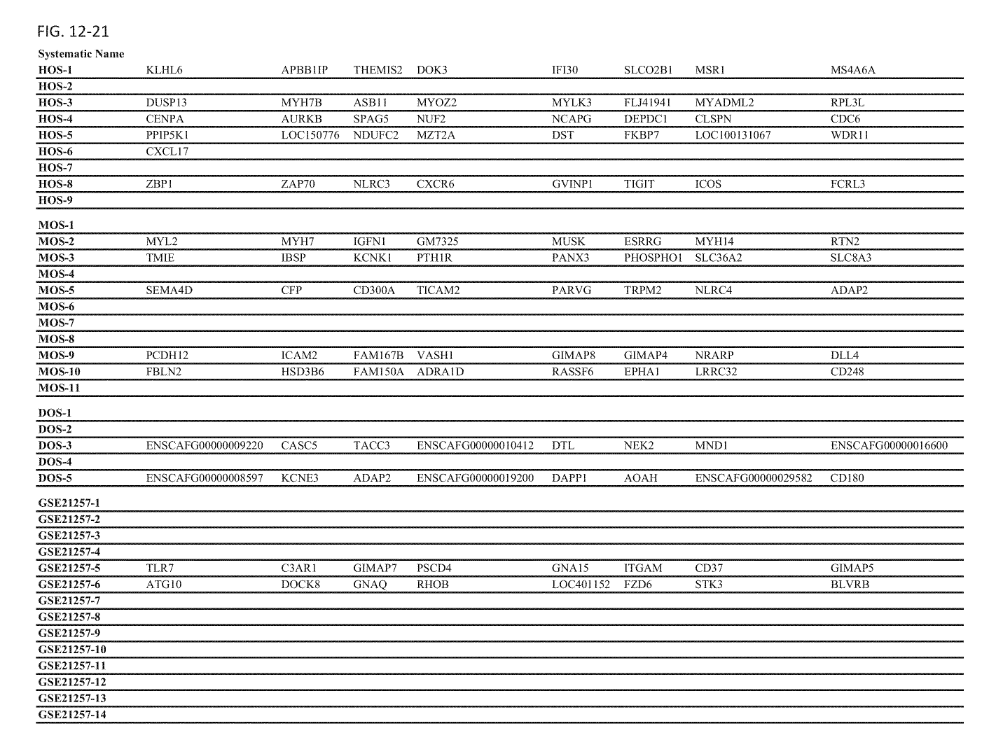

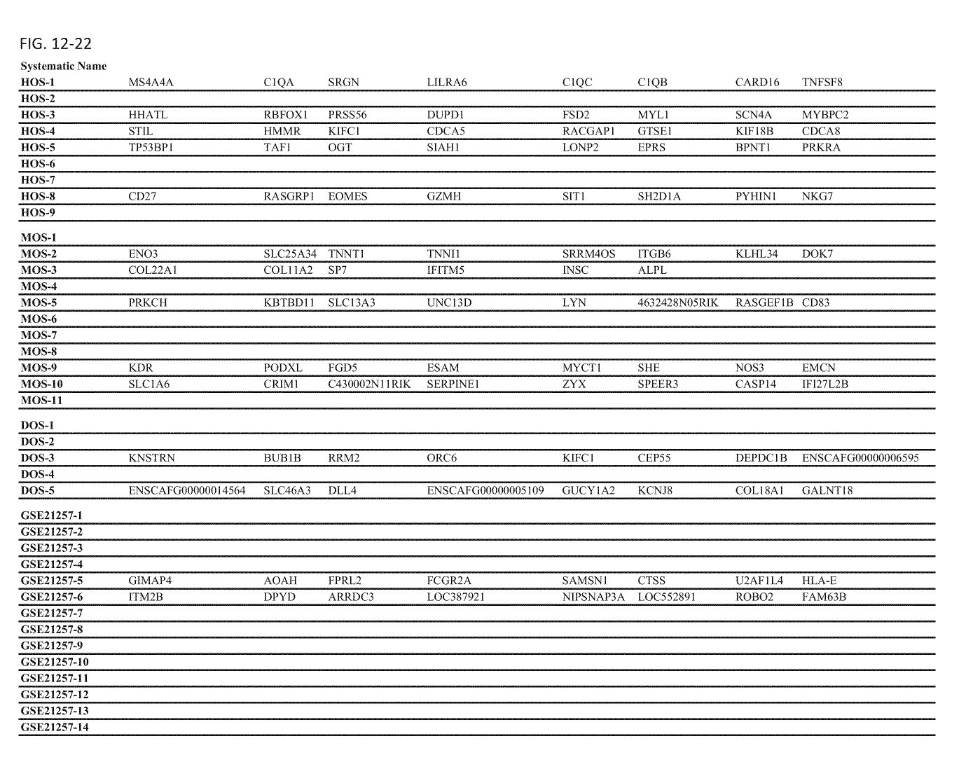

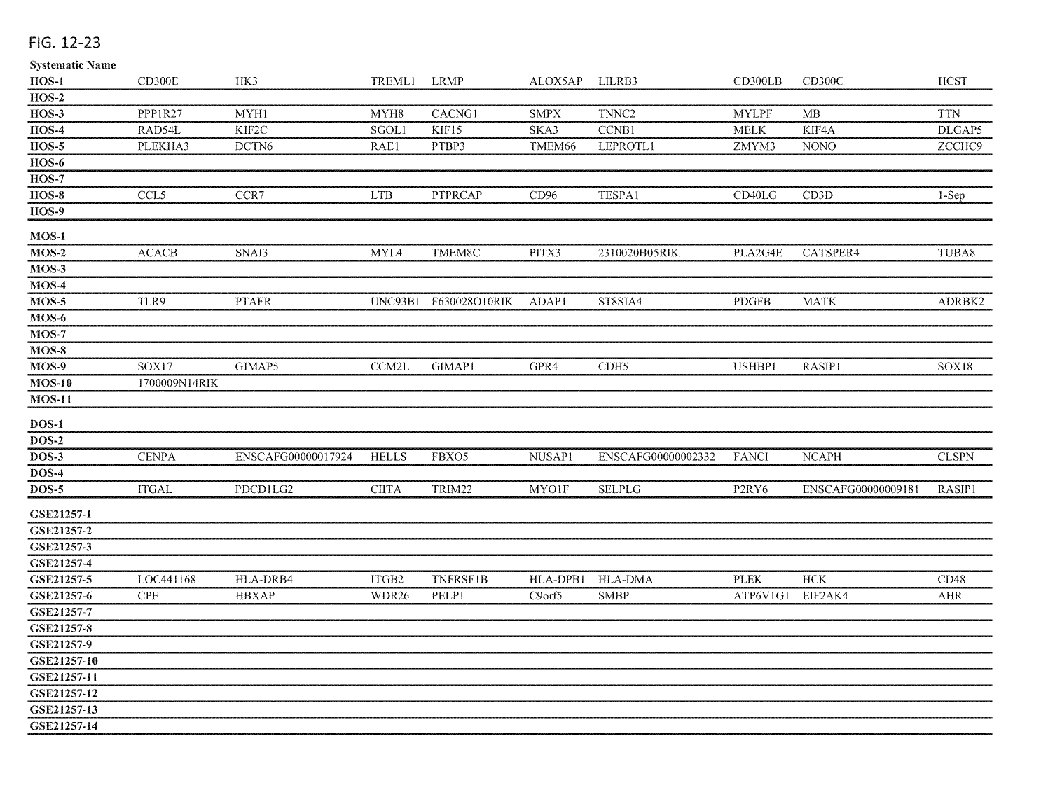

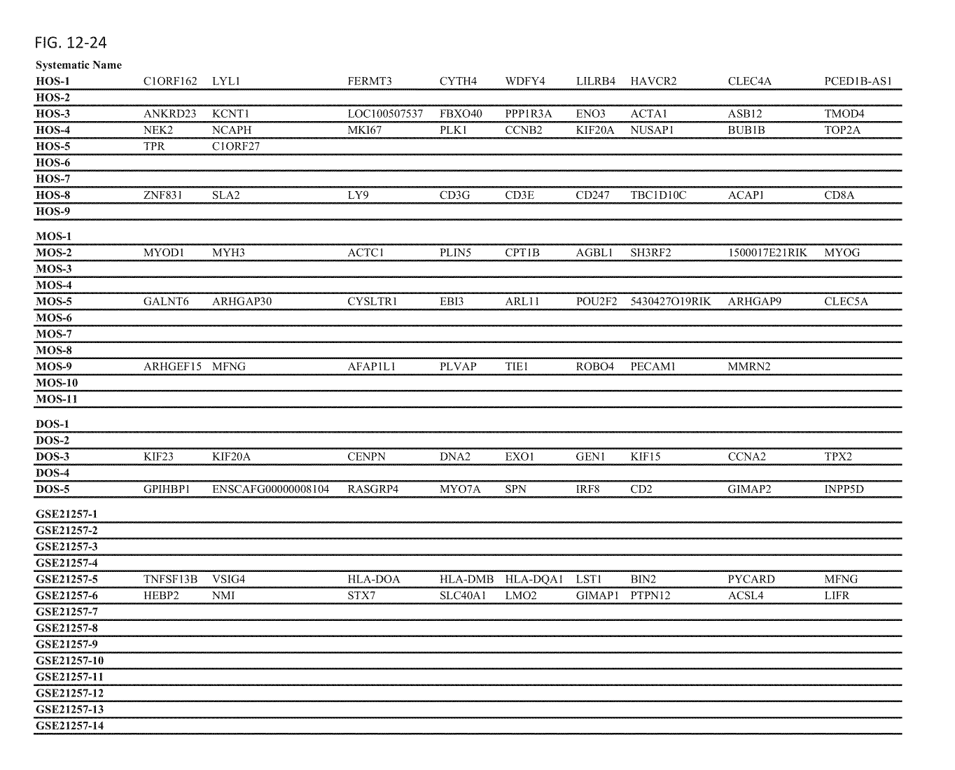

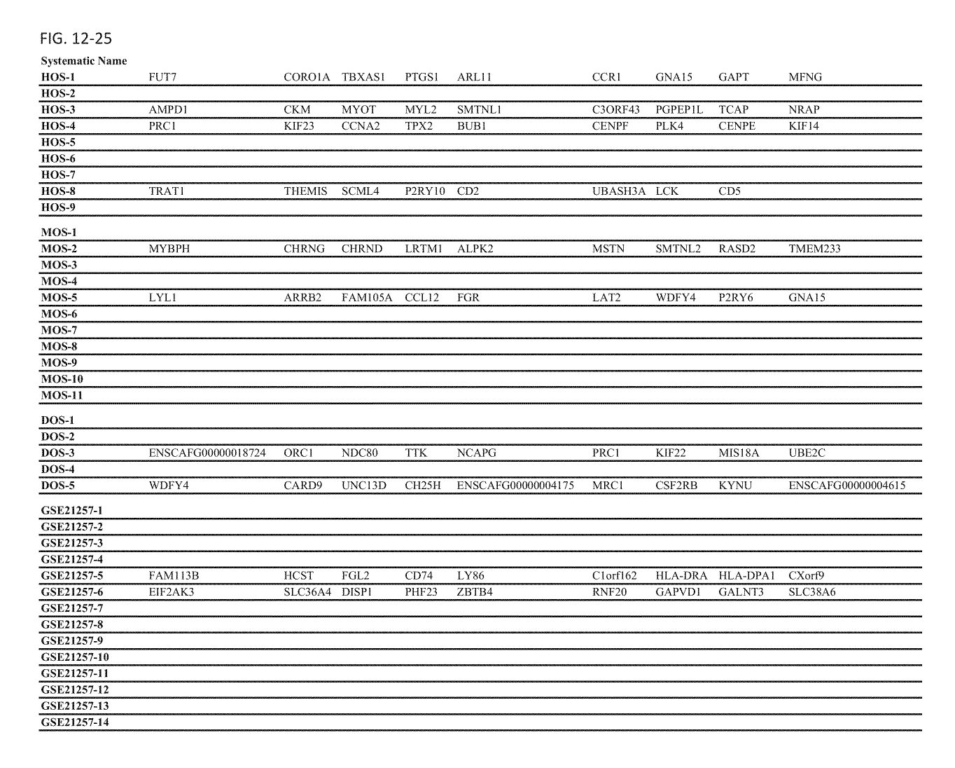

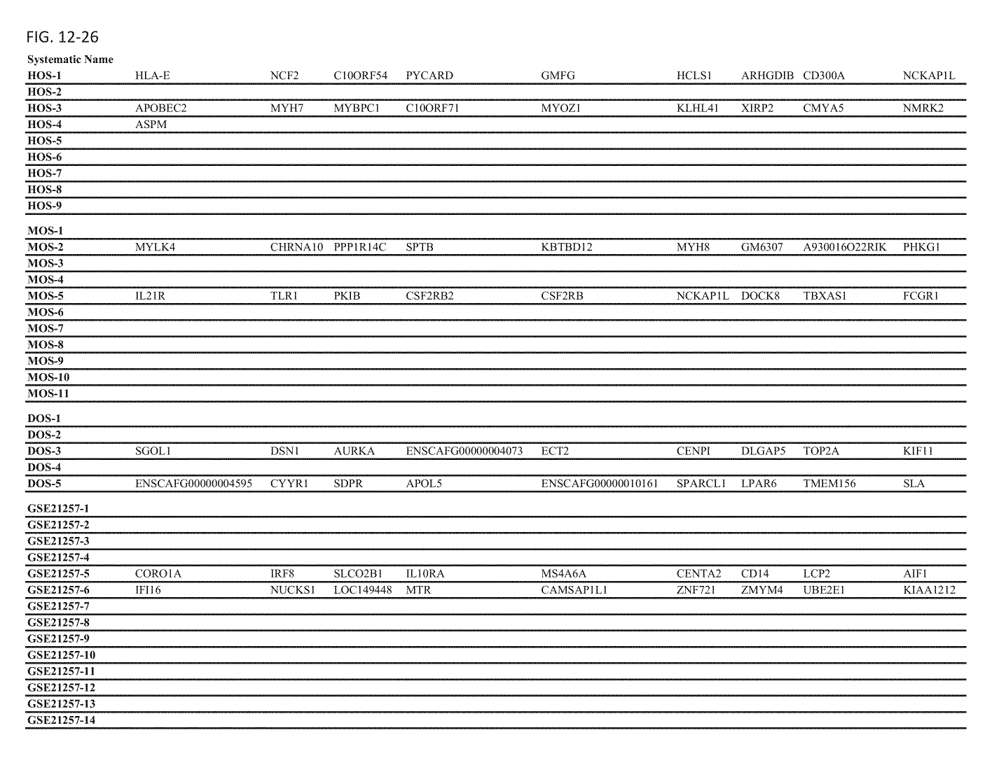

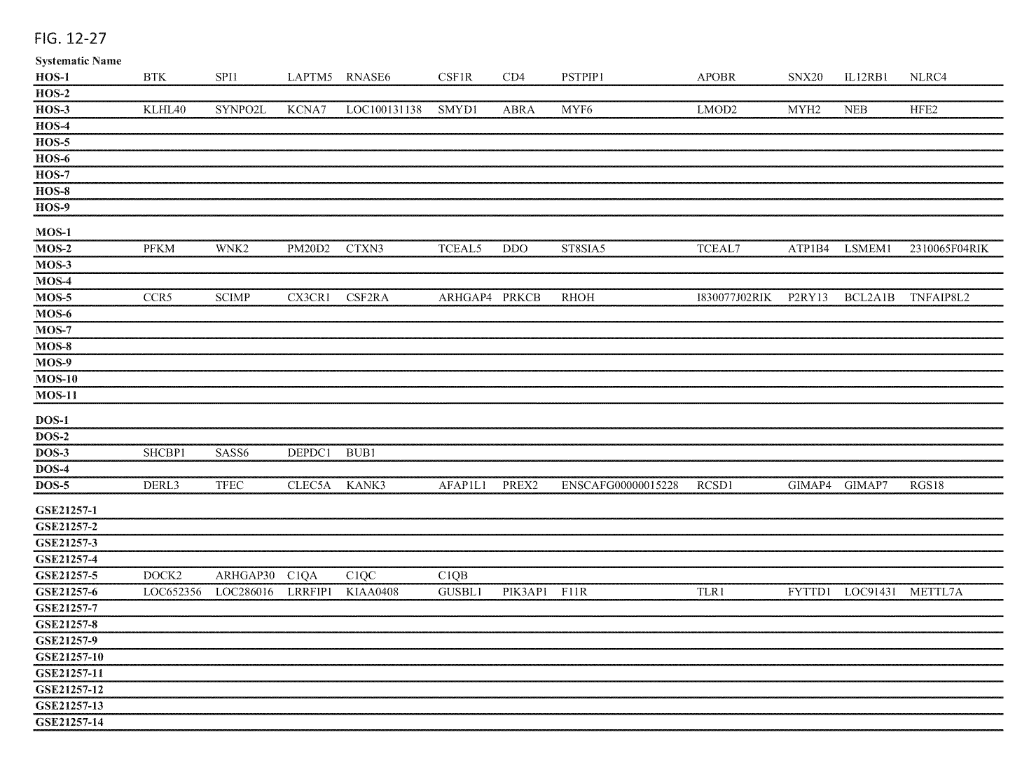

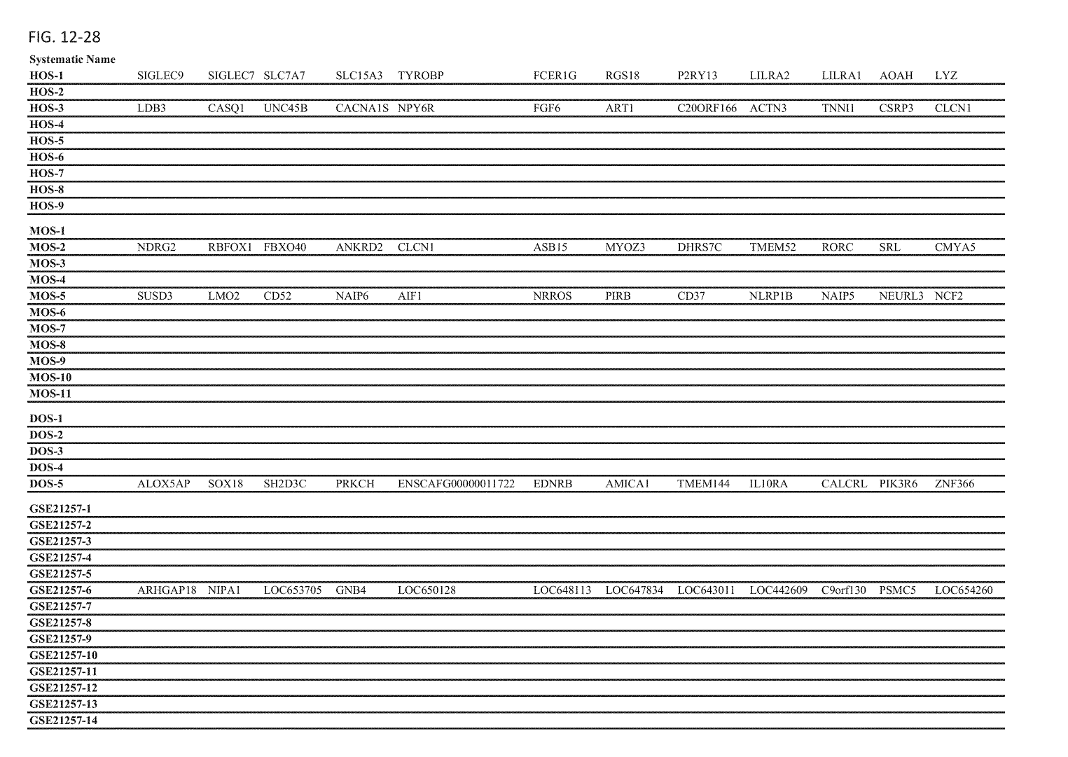

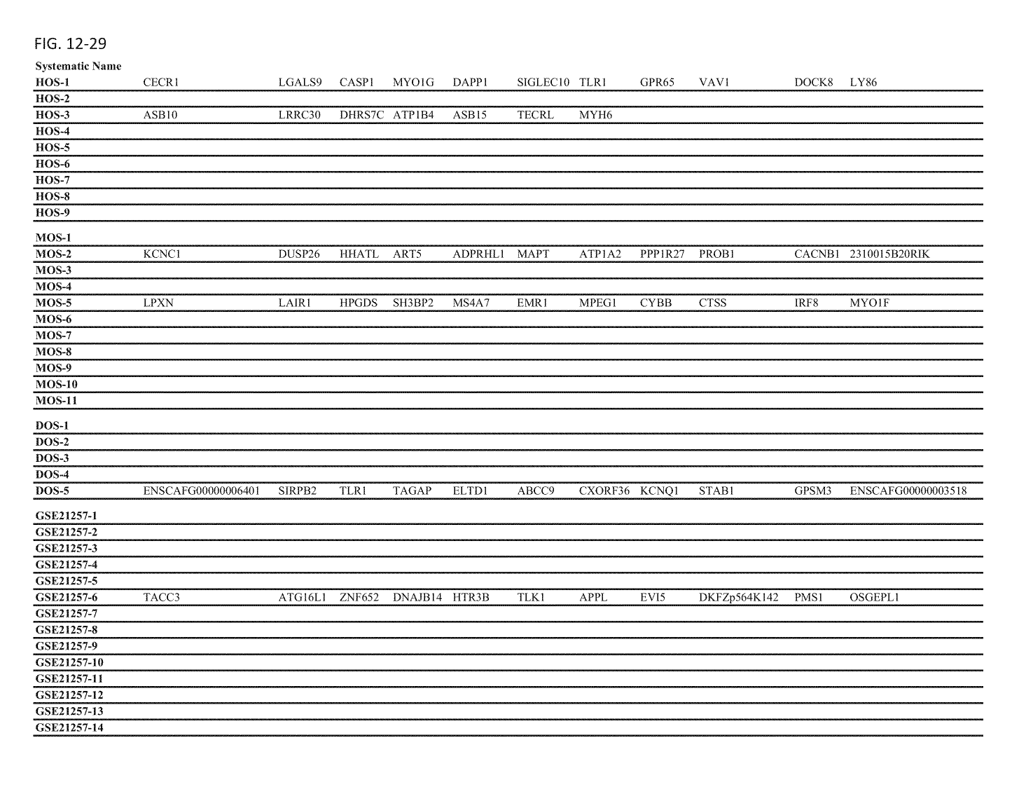

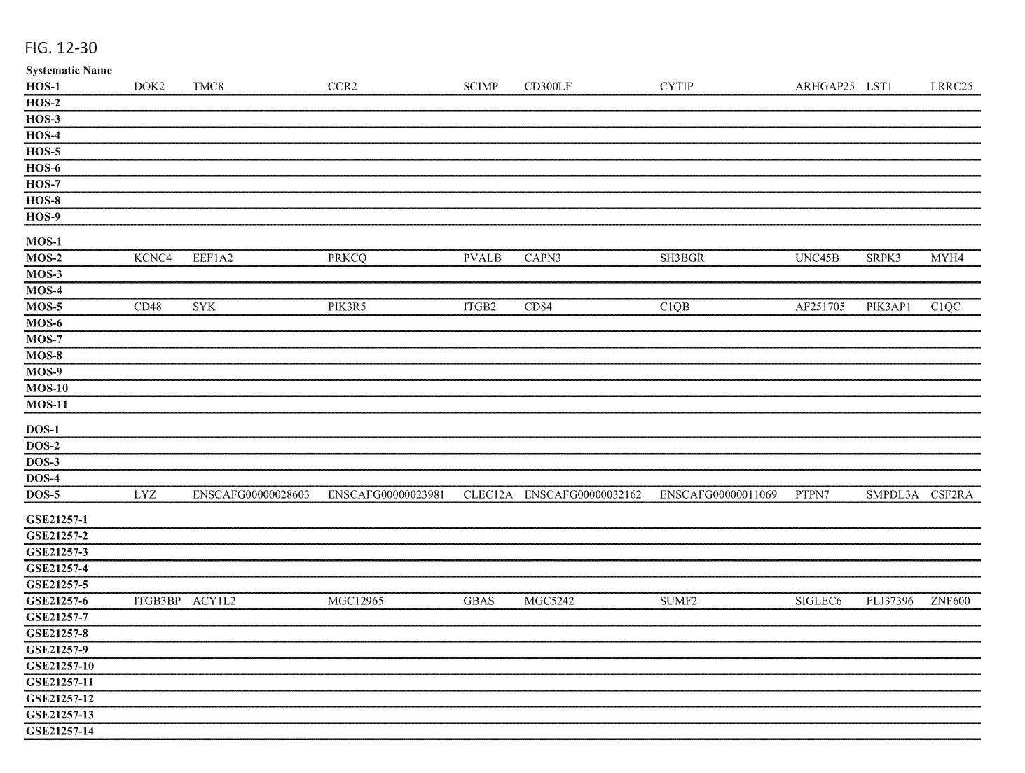

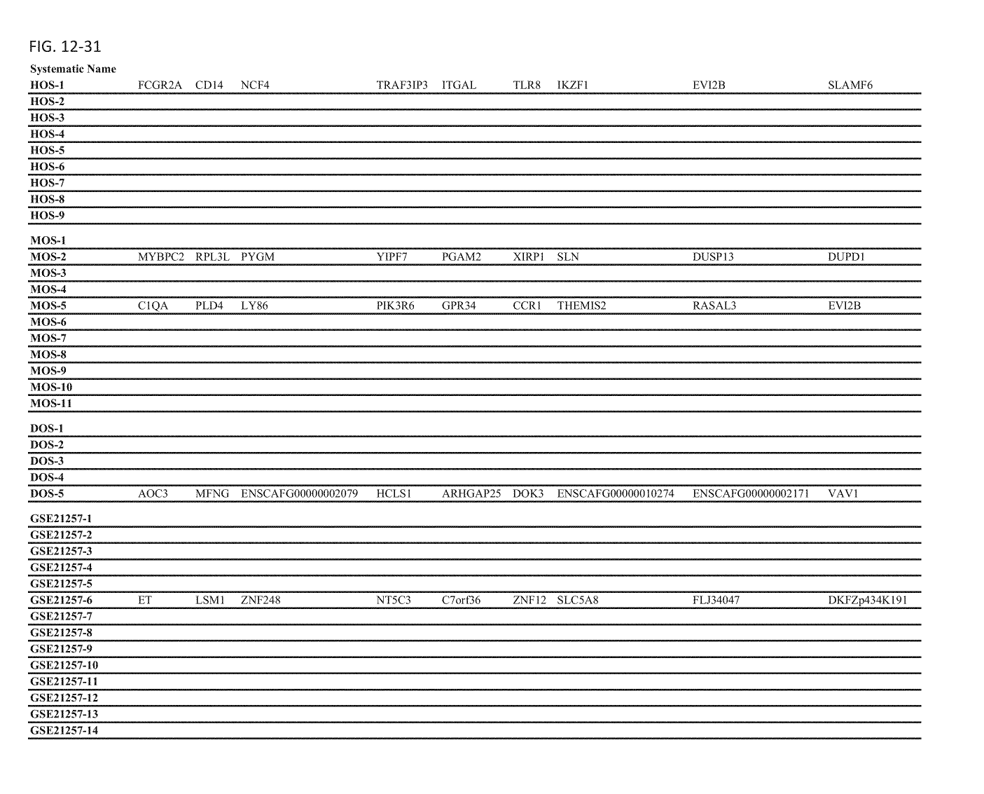

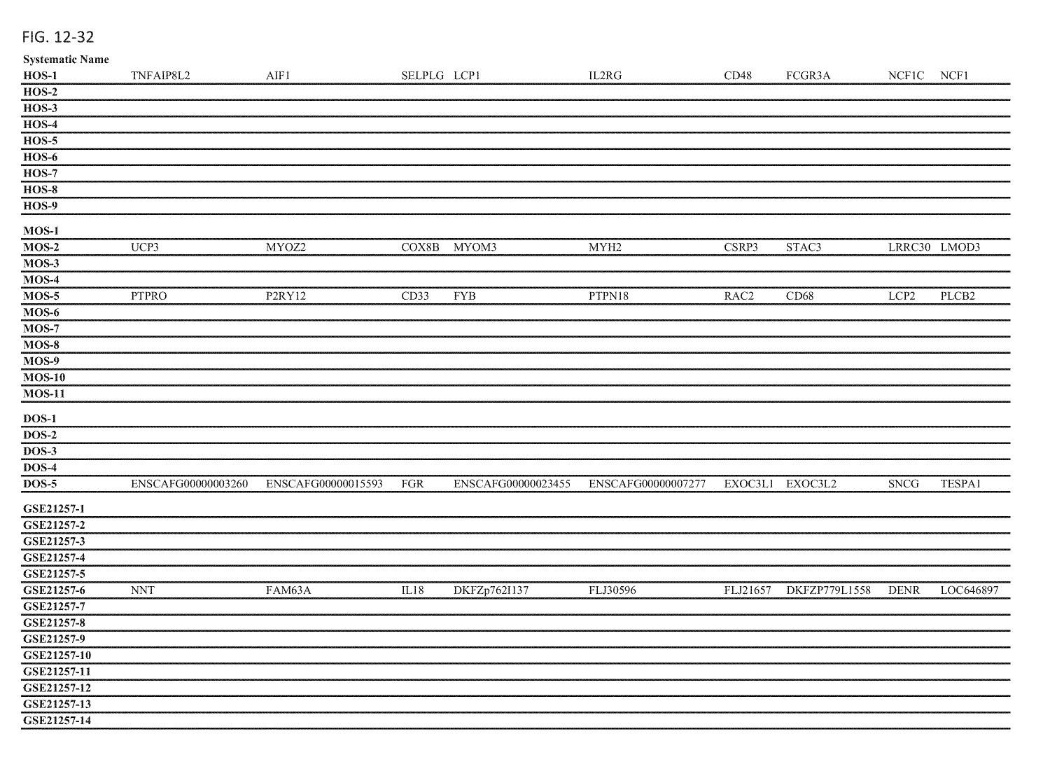

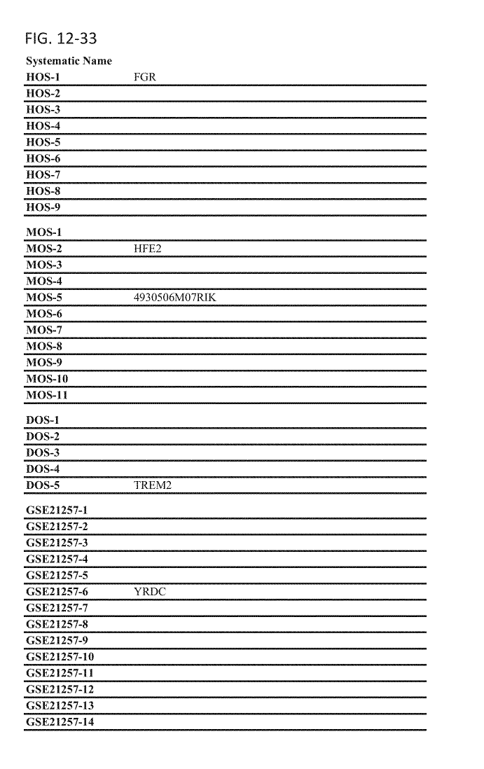

If the conserved tumor heterogeneity and association between GCESS and patient outcome revealed by this methodology are generally observable in osteosarcoma tumors, they should also be observable in older array hybridization based data. This was validated using GSE21257, an Illumina mRNA array dataset that contains genome wide expression data for 53 tumors from patients with known outcome data, including survival and metastasis (Buddingh et al., 2011. Clin Cancer Res 17(8):2110-2119). Following a strategy similar to the one used for the RNA-Seq data, 14 highly correlated gene clusters were identified. Gene overlap analyses comparing clusters derived from array and RNA-Seq data sets first identified four gene clusters from the array, which corresponded to the RNA-Seq immune cell clusters, and two array clusters, which corresponded to the RNA-Seq cell cycle cluster (FIG. 5A-B). Subsequent gene overlap analyses identified gene clusters from the array that corresponded to the RNA-Seq defined Immune-1, Immune-2, Cell Cycle and muscle transcript clusters (FIG. 11A). KM survival analyses using sample GCESSs generated from each of these array gene clusters again showed that patients whose tumors had low immune cell GCESSs were more likely to succumb to osteosarcoma. This result was independently observable for each of the four immune-cell-related array gene clusters (FIG. 5C; FIG. 11B-D).

A KM-analysis examining the time to metastasis was also performed. Low immune cell GCESSs or high cell cycle GCESSs were strongly associated with faster progression to metastasis (p=0.0001 and p=0.02 respectively). These results independently validate the findings obtained from the HOS2 dataset as well as the general utility of the methodology described independent of experimental platform.

Low Immune-2 GCESS Associated with Metastasis in Human and Mouse Samples.

Following necropsy, many of the mice had observable metastases. Scores from samples with Metastases had lower Immune-2 scores than samples where metastatic tumors were not observed. In both Human datasets, Immune-2 scores were lower in samples where Metastases were present at diagnosis. This difference became significant when patients with metastasis in less than one year were combined with patients where metastasis was observed at diagnosis. The significance became even stronger when patients which showed metastasis at any point were compared to patients where metastasis was not observed. These findings indicate that the Immune-2 score has prognostic potential for determining the likelihood of a tumor metastasizing in human patients (FIG. 11E).

OS-Derived Cell Cycle and Immune Cell GCESSs Correlate with Poor Outcomes Across Multiple Tumor Types

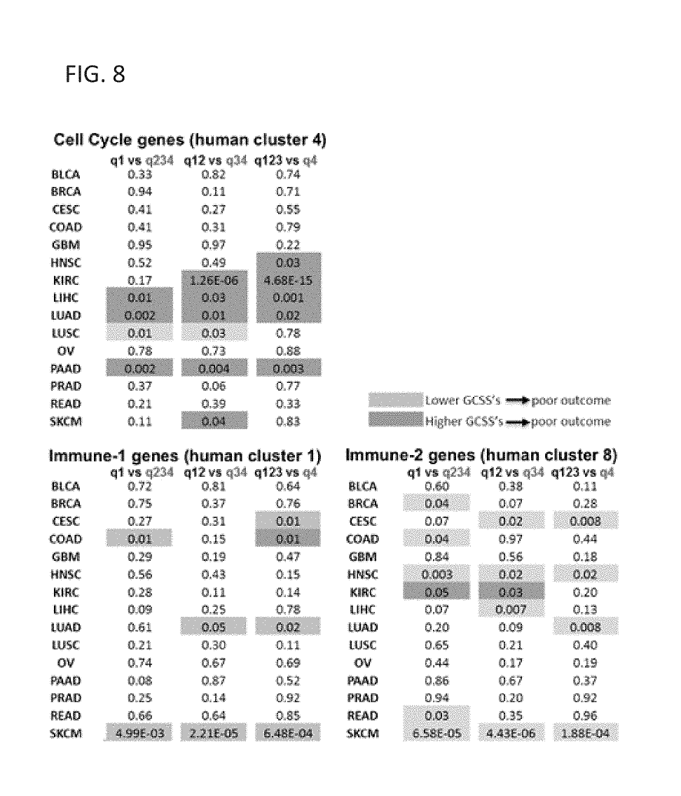

To determine if increased cell cycle transcripts and decreased immune cell transcripts are each also associated with poor outcome across different tumor types, GCESSs were calculated for each of the TCGA tumor datasets (using the genes comprising the respective human osteosarcoma clusters to identify each tumor's cell cycle and immune cluster), which were then subjected to KM-survival analysis. High cell cycle GCESSs were significantly associated with poor outcomes in Kidney Renal Clear Cell Carcinoma (KIRC), Liver Hepatocellular Carcinoma (LIHC), Lung Adenocarcinoma (LUAD), and Pancreatic adenocarcinoma (PAAD), Head and Neck Squamous Cell Carcinoma (HNSC), and Cutaneous Melanoma (SKCM) (FIG. 8). Low immune cell GCESSs from Human osteosarcoma gene cluster 1 were significantly associated with poor outcomes in SKCM, LUAD, COAD, and CESC (FIG. 8). Low immune cell GCESSs derived from Human osteosarcoma gene cluster 8 were significantly associated with poor outcomes in SKCM, HNSC, LUAD, LIHC, COAD, CESC and Breast Invasive Carcinoma (BRCA). These results indicate that the survival associations between cell cycle and immune transcript expression levels observed in osteosarcoma are also present in a wide range of tumor types and that the GCESS methodology is capable of observing these associations in datasets where improved sample power exists.

RNA-Seq expression data were downloaded for additional tumors from TCGA. Using a similar methodology, GCESSs were calculated for each of the TCGA tumor datasets (using the genes comprising the respective human osteosarcoma clusters to identify each tumor's cell cycle and immune cell clusters) and KM-analyses were used to determine correlation with poor survival outcomes. (A) High cell cycle GCESSs (based on human osteosarcoma cluster-4) were significantly associated (in red) with poor outcomes in KIRC, LIHC, LUAD, and PAAD. (B) Low immune cell GCESSs (based on human osteosarcoma cluster-1 (Monocyte enriched)) were significantly associated (in grey) with poor outcomes in CESC, COAD, LUAD, and SKCM. (C) Low immune cell GCESSs (based on human osteosarcoma cluster-8 (T-cell enriched)) were significantly associated (in grey) with poor outcomes in BRCA, CESC, COAD, HNSC, LIHC, LUAD, rectum adenocarcinoma (READ), and SKCM.

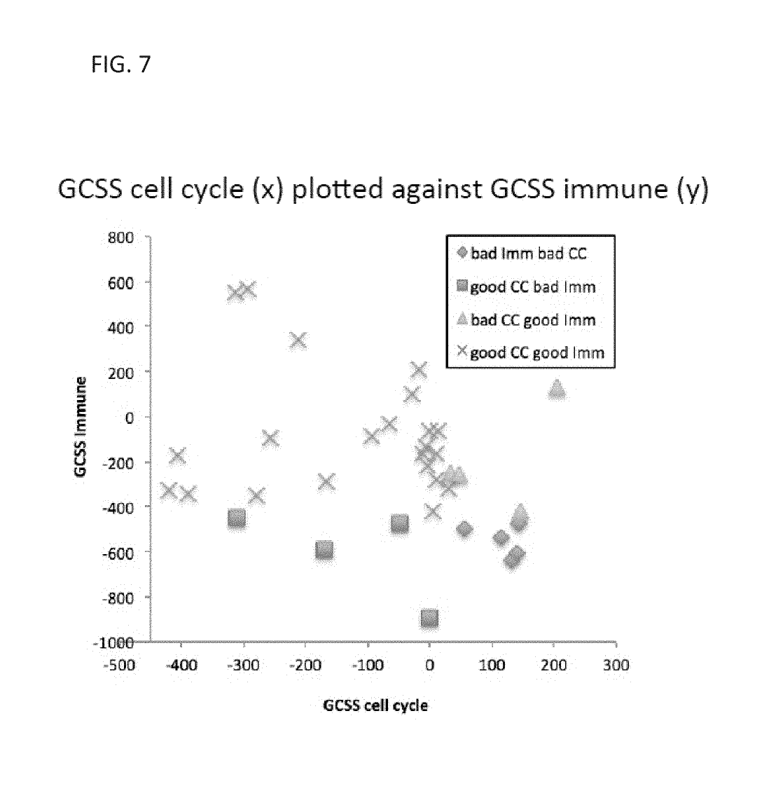

Thus, many diverse genetic events, including a catalog of rare events, have been reported to lead to osteosarcoma formation, progression, and/or metastasis. Loss of immune cell infiltration and increased levels of cell cycle transcripts are two specific transcriptional prognostic biomarkers for metastasis and overall poor outcome of osteosarcoma patients. These transcriptional signatures also had prognostic utility across many types of human tumors, suggesting they are common transcriptional markers of pathological progression. Further, these two transcriptional patterns appear to be independent (FIG. 7).

Tumor transcription can be conceptualized as resulting from loss of control of a series of independent transcriptional modules (gene clusters). The GCESS technique described in this disclosure generates a single meaningful value for each module in a tumor. The GCESS value can then be used for phenotype association discovery, thereby minimizing the multiple testing risks in datasets underpowered for meaningful genome-wide association analyses. These types of errors are clearly described for SNP association studies but remain prevalent in genome-wide studies utilizing large numbers of parallel analyses. Empirical testing of single gene based strategies to associate tumor transcript level with outcome revealed that for every 20 random transcripts tested, one false positive prognostic transcript was likely to be identified (using an uncorrected p<0.05). If 1,000 transcripts were tested, then this would result in 50 likely false-positives. Many genome-wide analyses of RNA-Seq or array data routinely involve testing thousands of transcripts, which would potentially result in hundreds of false positives. This may explain why many transcription-based prognostic tests fail to be reproducible in independent cohorts.

Increased levels of cell cycle/mitosis transcripts are observed in cell lines generated from osteosarcoma tumors from patients with worse outcomes. This disclosure describes the extension of that observation to the osteosarcoma tumor itself and provides a novel methodology for identifying transcriptionally related gene clusters, even if they are not the primary component present in the dataset, and testing their association with a variety of outcomes while minimizing errors from multiple testing. These results are highly consistent with the CINSARC signature (Complexity INdex in SARComas) predictive of metastasis free survival. Many of the genes identified encode for proteins involved in cell cycle/mitosis, cytokinesis, mitotic checkpoint, and DNA damage repair.

Also, cell-cycle-mediated events have more prognostic potential for dog disease progression compared to human disease. This may be due to differences in therapeutic regimens and overall commitment to therapy. Another potential confounding factor is the general characteristic of dog osteosarcoma to progress much faster than typically observed in either normal or late-onset human osteosarcoma. Thus, dogs with osteosarcoma may not survive long enough for the role of the immune cells to become observable. In specific dog breeds, the immune system may also have a reduced capability to recognize and respond to tumor cells.

Transcriptional profiles derived from grossly dissected tumors have variable types and quantities of cells present, including stromal and immune cells in addition to cancer cells. The GCESS technique described herein provides a straightforward method to indicate the relative abundance of immune cells in the tumor tissue sample, which can also be generally applied to any component present in a transcriptional dataset. Results from the GCESS method were compared to a previously published tool (ESTIMATE; Yoshihara et al., 2013. Nature Comm 4), which infers tumor purity. In naturally occurring human and dog osteosarcoma tumor samples there was a strong correlation between the immune cell GCESS values and the ESTIMATE immune scores.

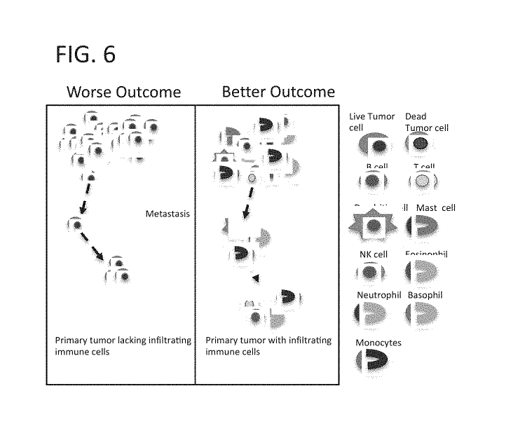

As a genomically chaotic disease, conventional wisdom suggests osteosarcoma must provide an abundance of antigens that typically would result in increased recognition by the host immune system, yet somehow osteosarcoma tumors and their metastases have proven capable of escaping immune surveillance. The results reported herein reinforce the theory that immune cell infiltration and monitoring is a normal process in bone tissue, and suggests that disruption of this process in a subset of tumors is associated with increased tumor mortality and progression to metastasis (FIG. 6). Analyses of the TCGA data showed that the association between decreased immune cell GCESS and outcome was strongest in Cutaneous Melanoma (SKCM), a data set containing a large number of metastatic samples. Some melanoma patients respond to immune checkpoint blockade therapy, and the results reported herein indicate that this same approach may have clinical utility in osteosarcoma patients. Furthermore, these results support an association between absence of immune cells, metastasis, and clinical outcome. Where immune cell outcome associations were seen in the TCGA the effects were more significant using the T cell enriched cluster relative to the monocyte enriched cluster. On the contrary, in our osteosarcoma datasets the monocyte enriched gene cluster was more significant (lower p values) than the T cell enriched gene cluster.

Progression to metastasis may be a result of decreased immune response in osteosarcoma patients. Alternatively, there may be some mechanism by which the tumor itself suppresses the immune response and that mechanism might be necessary for disease progression.

Using multi-species datasets provides unique opportunities and insights to identify biologically meaningful gene signatures and to identify aspects of the immune response that can be manipulated therapeutically to improve the quality of life and outcomes of children with bone cancer, as well as patients with other sarcomas and solid tumors.

Thus, while described herein in the context of certain exemplary embodiments—e.g., in which the GCESSs are calculated in an osteosarcoma patient—GCESSs may be calculated using the cell cycle gene cluster and the immune cell gene clusters described herein for patients having other forms of cancer. Exemplary other cancers for which the methods described herein may be performed include cervical squamous cell carcinoma and endocervical adenocarcinoma (CESC), colon adenocarcinoma (COAD), glioblastoma (GBM), acute myeloid leukemia (LAML), prostate cancer (PRAD), thyroid (THCA) cancer, bladder urothelial carcinoma (BLCA), breast invasive carcinoma (BRCA), head and neck squamous cell carcinoma (HNSC), kidney renal clear cell carcinoma (KIRC), liver hepatocellular carcinoma (LIHC), lung adenocarcinoma (LUAD), lung squamous cell carcinoma (LUSC), ovarian serous cystadenocarcinoma (OV), pancreatic adenocarcinoma (PAAD), rectum adenocarcinoma (READ), and skin cutaneous melanoma (SKCM).

Once a sample has been analyzed, a medical profession can use the results to assess the likelihood and/or extent to which the subject from whom the sample was obtained is at risk for having an aggressive tumor. This may indicate to a medical profession a preferred course of treatment for the subject. For example, analysis of a sample that results in GCESS that indicates that the subject is at risk for having an aggressive tumor may lead a medical profession to recommend and/or initiate aggressive therapy that may include aggressive chemotherapy, aggressive radiation therapy, and/or surgery. Conversely, analysis of a sample that results in GCESS that indicates that the subject is at low risk for having an aggressive tumor may lead a medical profession to recommend and/or initiate less aggressive therapy that may be effective fro treating the tumor but subject the patient to less severe side effects.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### Examples

### Methods

### Biospecimen Collection and Processing

Human and dog biospecimens were collected from newly diagnosed osteosarcoma patients prior to treatment with cytotoxic chemotherapy drugs. Specimens were obtained under protocols approved by either the University of Minnesota's Institutional Review Board or Institutional Animal Care and Use Committee (protocol numbers 0802A27363, 1101A94713, 1312-31131A) or the University of Colorado Institutional Review Board or Institutional Animal Care and Use Committee (AMC 635040202, AMC 200201jm, AMC 2002141jm, 02905603(01)1F, COMIRB 06-1008).

### Human

Human patient osteosarcoma samples (n=44) and normal bone samples (n=3) were obtained from the University of Minnesota Biological Materials Procurement Network (UMN BioNet) or the Cooperative Human Tissue Network (CHTN), both of which follow standardized patient consent protocols. Samples had been de-identified and only a limited amount of patient information was provided. To identify if any of the samples came from the same patient we used OptiType, a precision HLA typing tool (Szolek et al., A, Schubert B, Mohr C, Sturm M, Feldhahn M, Kohlbacher O. OptiType: precision HLA typing from next-generation sequencing data. Bioinformatics (Oxford, England). 2014; 30(23):3310-6). Five pairs of matching human patient samples were identified using OptiType. For four of the five pairs, the provided age, sex, and race of the patients were also identical (the 5^{th} pair did not have complete metadata information). Saos-2, U-2 OS, MG-63, and 143B human osteosarcoma cells line purchased from American Type Culture Collection (Manassas, Va.) and authenticated by the University of Arizona Genetics Core using short tandem repeat profiling, as well as an osteoblast cell line, which was a gift from Dr. Richard G. Gorlick (Albert Einstein College of Medicine, New York, N.Y.), were also sequenced.

### Dog

Osteosarcoma samples (total n=31) were obtained from dogs with naturally occurring primary appendicular tumors, recruited between 1999 and 2012. Most of the samples were from Rottweilers and Golden Retrievers. Specimens were obtained with owner consent under approved protocols as previously described (Scott et al., 2011. Bone 49(3):356-367). Also included in this study were two cell lines (OSCA-8 and OSCA-78) derived from primary osteosarcoma tumor as previously described(20), and one dog osteoblast sample, CnOB, purchased from Cell Applications, Inc. (San Diego, Calif.). The OSCA cell lines are available for distribution through Kerafast, Inc. (Boston, Mass.).

### Mouse

Included in this study were 25 osteosarcoma samples from mice with somatic induction of Trp53^{R270H} expression in Osx1-expressing cells (Moriarity et al., 2015. Nat Genet 47(6):615-624), 67 samples from Sleeping Beauty transposon accelerated osteosarcoma in wildtype and Trp53^{R270H} mice (Temiz et al., 2016. Genome Res 26(1):119-129), and 11 cell lines established from mouse osteosarcoma tumors (Moriarity et al., 2015. Nat Genet 47(6):615-624).

RNA Extraction from Frozen Tumor Tissue and Cell Lines Isolation of total RNA from tissues and cell lines was performed according to the recommended protocol for AMBION TotalRNA kit (Thermo Fisher Scientific, Inc., Waltham, Mass.). Samples were quantified using fluorescence by RIBOGREEN dye (Thermo Fisher Scientific, Inc., Waltham, Mass.). RNA integrity was assessed using capillary electrophoresis with the 2100 BioAnalyzer system (Agilent Technologies, Santa Clara, Calif.) which generated an RNA Integrity Number (RIN). Samples with a RIN >6.5 were included in this study.

### RNA-Sequencing (RNA-Seq)

Paired end sequences (30-40 million reads per sample) were generated for dog, mouse, and human (tissue bank) samples at the University of Minnesota Genomics Center (UMGC) on a High Seq 2000 (Illumina, Inc., San Diego, Calif.) and delivered as FASTQ files.

RNA-Seq FASTQ files and outcome related metadata for 35 additional human osteosarcoma samples were obtained from dbGap:phs000699.v1.p1 (http://www.ncbi.nlm.nih.gov/gap) (Perry et al., 2014. Proceedings of the National Academy of Sciences USA 111(51):E5564-E5573). RNA-Seq files were also obtained from 25 additional human osteosarcoma samples available from previously published studies (Moriarity et al., 2015. Nat Genet 47(6):615-624; Man et al., 2004. BMC Cancer 4:45; Chen et al., 2014. Cell Reports 7(1):104-112).

FASTQ files from the HOS2 cohort were mapped to the reference genome and FPKM values were calculated using the same protocols as the samples in this study, while FASTQ files from the HOS3 cohort were mapped to the reference genome and RSEM expression values were calculated using the TCGA RNA-Sequencing protocol (Wang et al., 2010. Nucleic Acids Res 38(18):e178; Li B, Dewey C N, 2011. BMC Bioinformatics 12:323). In this study, the samples from both cohorts were used for calculating pairwise Pearson correlation coefficients and were included in the transcriptome analyses described below. The HOS2 cohort included survival data and information on presence of metastasis at diagnosis so this cohort was used for survival analysis.

GEO dataset series GSE21257, which consisted of genome-wide expression data of 53 human OS tumors produced from the Illumina human-6 v2.0 expression array was downloaded for this study. Patient outcome data, including survival and metastasis, was included in this study.

The Cancer Genome Atlas (TCGA) data portal, (http://tcga-data.nci.nih.gov/tcga) was used to download survival times, death events, and RNA-Seq data for 5582 tumors, summarized in Table 2.

TABLE 2

| | | | Metastasis at |
|---|---|---|---|
| Dataset | Tumors | Deaths | Diagnosis |
| DOS | 19 | 19 | |
| HOS | 35 | 17 | 17 |
| GSE212257 | 53 | 23 | 34 |
| BLCA_tumor-data.txt | 266 | 108 | |
| BRCA_tumor-data.txt | 1054 | 157 | |
| CESC_tumor-data.txt | 98 | 28 | |
| COAD_tumor-data.txt | 415 | 91 | |
| GBM_tumor-data.txt | 152 | 118 | |
| HNSC_tumor-data.txt | 499 | 211 | |
| KIRC_tumor-data.txt | 529 | 175 | |
| LIHC_tumor-data.txt | 194 | 89 | |
| LUAD_tumor-data.txt | 463 | 156 | |
| LUSC_tumor-data.txt | 476 | 201 | |
| OV_tumor-data.txt | 302 | 172 | |
| PAAD_tumor-data.txt | 180 | 93 | |
| PRAD_tumor-data.txt | 417 | 8 | |
| READ_tumor-data.txt | 157 | 21 | |
| SKCM_tumor-data.txt | 380 | 187 | |

### RNA-Seq Workflow

Initial quality control analysis of RNA-Seq data for each sample was performed using FastQC (version 0.11.2) (Babraham Institute, Cambridge, United Kingdom). Each sample was aligned using the Tophat aligner (version 2.0.13; Trapnell et al., 2009. Bioinformatics 25(9):1105-1111.). Samtools software (version 1.0_BCFTools_HTSLib) was used to sort and index the bam files (Li et al., 2009. Bioinformatics 25(16):2078-2079). Cuffquant (Cufflinks version 2.2.1) was used to generate transcript abundance files (options used include the multi-read-correct, max-bundle-frags <10000000> and mask-file (for genes <200 bp). Once all samples within each species were mapped and abundance estimate files were completed, Cuffnorm (Cufflinks version 2.2.1), was used to generate a table of Fragments Per Kilobase Of Exon Per Million Fragments Mapped (FPKM) values for genes within each sample (Trapnell et al., 2012. Nature Protocols 7(3):562-578).

### Reference Genomes

The University of California, Santa Cruz (UCSC) Genome Browser version hg19 (GRCh37 assembly) of the human reference genome and Ensembl Genes v70 were used for mapping human sequences (Hubbard et al., 2002. Nucleic Acids Res 30(1):38-41). Version canFam3 (Broad Institute v3.1) was used as the dog reference genome in this study (Hoeppner et al., 2014. PLoS ONE 9(3):e91172). The Broad Institute provided .bed files (personal communication) and from these a .gtf file was created. UCSC version mm10 (GRCm38, Genome Reference Consortium Mouse Build 38 (GCA_000001635.2)) was used as the mouse reference genome.

### Transcriptome Analyses

For each dataset, 0.1 was added to each FPKM value. 12,062 orthologous genes present in all RNA-Seq datasets were used for calculating pairwise Pearson's correlation values between all samples. Due to the high correlations between human osteosarcoma datasets from multiple sources, the human datasets were combined for further analyses (Chen et al., 2014. Cell Reports 7(1):104-12; Perry et al., 2014. Proceedings of the National Academy of Sciences USA 111(51):E5564-E5573). The 8,000 most variable genes across the full same-species datasets were identified for clustering by species (standard deviation cutoffs: human >0.93, mouse >0.72, and dog >0.79). Cluster 3.0 (C Clustering Library 1.52) was used to log₂ transform and gene-mean-center the data and then perform hierarchical average linkage clustering using the Pearson similarity metric. Clustering data were visualized in Java TreeView (version 1.1.6r4). OptiType, a precision HLA typing tool (Szolek et al., 2014. Bioinformatics 30(23):3310-3316) was used to identify human OS samples derived from the same patient.

### Identification of Strongly Conserved Clusters of Genes

Gene clusters with a dendrogram node correlation >0.60 and at least 60 individual genes were identified in each of the species datasets. The 0.6 Pearson correlation cutoff value was chosen, as it is a widely accepted conservative confidence threshold. The minimum cluster size of 60 was chosen to ensure that only larger transcriptional patterns were identified. Permuted and random datasets were used to show that these thresholds would not identify clusters in artificial datasets that do not contain meaningful transcription patterns. Gene clusters representing batch effects from the combination of different human samples were removed from further analyses.

### Generation of Control Datasets

For each species tumor dataset, a random dataset and a permuted dataset were generated as controls. Random datasets were generated by randomly selecting values between −2 and 2 to replace the actual (mean-centered) values. Permuted datasets were generated by randomly reordering the values for each gene.

### Gene Cluster Expression Summary Score (GCESS) Calculation

The GCESS is defined as the sum of expression values (log₂-transformed and mean centered) of all genes in a particular defined cluster for a single sample.

### Pathway Analysis

The Ingenuity Pathway Analysis (IPA) suite (Qiagen, Redwood City, Calif.) was used to identify pathways associated with gene clusters.

### Statistics

Statistical significance was calculated using the log-rank test or by Fisher's exact test depending on analysis and a p<0.05 was considered significant. Survival plots were generated using the ‘survival’ package in RStudio (Version 0.98.1103; RStudio, Boston, Mass.) (Therneau T M, Grambsch P M., 2000. Modeling Survival Data: Extending the Cox Model. New York: Springer; Therneau T M, 2015. A Package for Survival Analysis in S. version 2.38, Available from: http://CRAN.R-project.org/package=survival). The GCESS values were used to rank the tumors into quartile groups and the quartile groups were systematically tested for association with outcome.

### Additional Data

TCGA data was obtained from The Cancer Genome Atlas data portal, (http://tcga-data.nci.nih.gov/tcga). Hybridization based array and outcome data for 53 human osteosarcoma patients was obtained from GEO dataset GSE21257 (http://www.ncbi.nlm.nih.gov/geo/; Buddingh et al., 2011 Clin Cancer Res 17(8):2110-2119).

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A method for detecting aggressive tumor behavior, the method comprising:
analyzing a tumor sample from the subject for expression of transcripts from coding regions of the cell cycle gene cluster, the immune-1 gene cluster, or the immune-2 gene cluster;
computing a sum of log₂-transformed mean-centered expression values, thereby generating a Gene Cluster Expression Summary Score (GCESS) for the sample; and
detecting a tumor with aggressive behavior when:
the cell cycle gene cluster is analyzed and the sample GCESS is greater than 0; or
the immune-1 gene cluster or the immune-2 gene cluster is analyzed and the sample GCESS is less than 0.

2. The method of claim 1 wherein the immune-1 gene cluster and the immune-2 gene cluster is analyzed and the sample GCESS is less than 0.

3. The method of claim 1 wherein aggressive tumor behavior comprises rapid local tumor growth, rapid progression to metastasis, or below average response to standard of care treatment.

4. The method of claim 1 wherein the tumor comprises osteosarcoma (OS), include cervical squamous cell carcinoma and endocervical adenocarcinoma (CESC), colon adenocarcinoma (COAD), glioblastoma (GBM), acute myeloid leukemia (LAML), prostate cancer (PRAD), thyroid (THCA) cancer, bladder urothelial carcinoma (BLCA), breast invasive carcinoma (BRCA), head and neck squamous cell carcinoma (HNSC), kidney renal clear cell carcinoma (KIRC), liver hepatocellular carcinoma (LIHC), lung adenocarcinoma (LUAD), lung squamous cell carcinoma (LUSC), ovarian serous cystadenocarcinoma (OV), pancreatic adenocarcinoma (PAAD), rectum adenocarcinoma (READ), and skin cutaneous melanoma (SKCM).

5. The method of claim 1 further comprising administering to the subject treatment for a tumor showing aggressive behavior.

6. A method for detecting relative risk of metastasis in a patient diagnosed with a tumor, the method comprising:
analyzing a tumor sample from the patient for expression of transcripts from coding regions of the cell cycle gene cluster, the immune-1 gene cluster, or the immune-2 gene cluster;
computing a sum of log₂-transformed mean-centered expression values, thereby generating a Gene Cluster Expression Summary Score (GCESS) for the sample; and
detecting above average risk of metastasis when:
the cell cycle gene cluster is analyzed and the sample GCESS is greater than 0; or
the immune-1 gene cluster or the immune-2 gene cluster is analyzed and the sample GCESS is less than 0.

7. The method of claim 6 wherein the immune-1 gene cluster and the immune-2 gene cluster is analyzed and the sample GCESS is less than 0.

8. The method of claim 6 wherein the tumor comprises osteosarcoma (OS), include cervical squamous cell carcinoma and endocervical adenocarcinoma (CESC), colon adenocarcinoma (COAD), glioblastoma (GBM), acute myeloid leukemia (LAML), prostate cancer (PRAD), thyroid (THCA) cancer, bladder urothelial carcinoma (BLCA), breast invasive carcinoma (BRCA), head and neck squamous cell carcinoma (HNSC), kidney renal clear cell carcinoma (KIRC), liver hepatocellular carcinoma (LIHC), lung adenocarcinoma (LUAD), lung squamous cell carcinoma (LUSC), ovarian serous cystadenocarcinoma (OV), pancreatic adenocarcinoma (PAAD), rectum adenocarcinoma (READ), and skin cutaneous melanoma (SKCM).

9. The method of claim 6 further comprising administering to the subject treatment for a metastatic tumor.
